SEQUENCE LISTING (72 sequences)

SEQ ID NO: 1 - DNA, 20 bp, Artificial sequence
  Single strand DNA oligonucleotide
gctttcagcttccctgaaca

SEQ ID NO: 2 - DNA, 20 bp, Artificial sequence
  Single strand DNA oligonucleotide
ggagctgcttcacaggattc

SEQ ID NO: 3 - DNA, 19 bp, Artificial sequence
  Single strand DNA oligonucleotide
gccgagagctacacgttca

SEQ ID NO: 4 - DNA, 19 bp, Artificial sequence
  Single strand DNA oligonucleotide
gaccggtgcaatcttcaaa

SEQ ID NO: 5 - DNA, 18 bp, Artificial sequence
  Single strand DNA oligonucleotide
ctccatgtgccggatagc

SEQ ID NO: 6 - DNA, 22 bp, Artificial sequence
  Single strand DNA oligonucleotide
cgatttcaccagaagcctctac

SEQ ID NO: 7 - DNA, 20 bp, Artificial sequence
  Single strand DNA oligonucleotide
ccgaagtcagttccttgtgg

SEQ ID NO: 8 - DNA, 19 bp, Artificial sequence
  Single strand DNA oligonucleotide
catgggttctgacggacat

SEQ ID NO: 9 - DNA, 22 bp, Artificial sequence
  Single strand DNA oligonucleotide
tttgacttgcatgaagagaagc

SEQ ID NO: 10 - DNA, 22 bp, Artificial sequence
  Single strand DNA oligonucleotide
agctgtctctgaaagggacatt

SEQ ID NO: 11 - DNA, 24 bp, Artificial sequence
  Single strand DNA oligonucleotide
aggatgagttctttagctttgctt

SEQ ID NO: 12 - DNA, 21 bp, Artificial sequence
  Single strand DNA oligonucleotide
aataggctgccttcaacacaa

SEQ ID NO: 13 - DNA, 20 bp, Artificial sequence
  Single strand DNA oligonucleotide
cctatgaccccagtcaatgc

SEQ ID NO: 14 - DNA, 20 bp, Artificial sequence
  Single strand DNA oligonucleotide
acagcaaagtagggcacctc

SEQ ID NO: 15 - DNA, 18 bp, Artificial sequence
  Single strand DNA oligonucleotide
gcagccgtccttgtaacc

SEQ ID NO: 16 - DNA, 20 bp, Artificial sequence
  Single strand DNA oligonucleotide
ggctggctgtaaatcacctt

SEQ ID NO: 17 - DNA, 19 bp, Artificial sequence
  Single strand DNA oligonucleotide
gtacaaggcagctggcaac

SEQ ID NO: 18 - DNA, 21 bp, Artificial sequence
  Single strand DNA oligonucleotide
tgggaagctgagaatgatctg

SEQ ID NO: 19 - DNA, 19 bp, Artificial sequence
  Single strand DNA oligonucleotide
tgcctaagatgcccgactt

SEQ ID NO: 20 - DNA, 18 bp, Artificial sequence
  Single strand DNA oligonucleotide
agctgctggctggtgaag

SEQ ID NO: 21 - DNA, 23 bp, Artificial sequence
  Single strand DNA oligonucleotide
ttaccaaattgtagaggctttcg

SEQ ID NO: 22 - DNA, 24 bp, Artificial sequence
  Single strand DNA oligonucleotide
ccctgcctctatacactcactacc

SEQ ID NO: 23 - DNA, 20 bp, Artificial sequence
  Single strand DNA oligonucleotide
acccctcggtcttaggaaaa

SEQ ID NO: 24 - DNA, 19 bp, Artificial sequence
  Single strand DNA oligonucleotide
ttgctgatctctgccgtgt

SEQ ID NO: 25 - DNA, 22 bp, Artificial sequence
  Single strand DNA oligonucleotide
aggcttcttctacacacccaag

SEQ ID NO: 26 - DNA, 18 bp, Artificial sequence
  Single strand DNA oligonucleotide
cacaatgccacgcttctg

SEQ ID NO: 27 - DNA, 20 bp, Artificial sequence
  Single strand DNA oligonucleotide
caaccaggatggatttcagg

SEQ ID NO: 28 - DNA, 20 bp, Artificial sequence
  Single strand DNA oligonucleotide
cagtcccagcttctctccat

SEQ ID NO: 29 - DNA, 21 bp, Artificial sequence
  Single strand DNA oligonucleotide
ggaagatgggagacaaagtcc

SEQ ID NO: 30 - DNA, 20 bp, Artificial sequence
  Single strand DNA oligonucleotide
actgaagcagtttgggcact

SEQ ID NO: 31 - DNA, 19 bp, Artificial sequence
  Single strand DNA oligonucleotide
agcgagaagtgccaactcc

SEQ ID NO: 32 - DNA, 20 bp, Artificial sequence
  Single strand DNA oligonucleotide
ttgtacaggtcccgctcttt

SEQ ID NO: 33 - DNA, 20 bp, Artificial sequence
  Single strand DNA oligonucleotide
agcaccatcaacctccaaaa

SEQ ID NO: 34 - DNA, 20 bp, Artificial sequence
  Single strand DNA oligonucleotide
ggcgatgatgagtccttcag

SEQ ID NO: 35 - DNA, 24 bp, Artificial sequence
  Single strand DNA oligonucleotide
ctgctcaggacctactaacaacaa

SEQ ID NO: 36 - DNA, 19 bp, Artificial sequence
  Single strand DNA oligonucleotide
gtccagcttggaggacctt

SEQ ID NO: 37 - DNA, 19 bp, Artificial sequence
  Single strand DNA oligonucleotide
cgagggccttcagtactcc

SEQ ID NO: 38 - DNA, 19 bp, Artificial sequence
  Single strand DNA oligonucleotide
ggggacttggagcttgagt

SEQ ID NO: 39 - DNA, 20 bp, Artificial sequence
  Single strand DNA oligonucleotide
cgttggggatgacagagagt

SEQ ID NO: 40 - DNA, 20 bp, Artificial sequence
  Single strand DNA oligonucleotide
cgagtcctgcttcttcttgg

SEQ ID NO: 41 - DNA, 18 bp, Artificial sequence
  Single strand DNA oligonucleotide
aggaaccgagagccaggt

SEQ ID NO: 42 - DNA, 20 bp, Artificial sequence
  Single strand DNA oligonucleotide
tgagcaatgccctttagctt

SEQ ID NO: 43 - DNA, 23 bp, Artificial sequence
  Single strand DNA oligonucleotide
tctagtgcccatttactctggac

SEQ ID NO: 44 - DNA, 18 bp, Artificial sequence
  Single strand DNA oligonucleotide
gcaggtggacaggagcag

SEQ ID NO: 45 - DNA, 19 bp, Artificial sequence
  Single strand DNA oligonucleotide
cacatccctgccctcctac

SEQ ID NO: 46 - DNA, 21 bp, Artificial sequence
  Single strand DNA oligonucleotide
gaagagccggcttctctaaac

SEQ ID NO: 47 - DNA, 20 bp, Artificial sequence
  Single strand DNA oligonucleotide
accccagactccgtcagttt

SEQ ID NO: 48 - DNA, 20 bp, Artificial sequence
  Single strand DNA oligonucleotide
acagcagctctgccaagaag

SEQ ID NO: 49 - DNA, 21 bp, Artificial sequence
  Single strand DNA oligonucleotide
gggggaatggaccttgtatag

SEQ ID NO: 50 - DNA, 20 bp, Artificial sequence
  Single strand DNA oligonucleotide
gcaaagctcctaccgtacca

SEQ ID NO: 51 - DNA, 24 bp, Artificial sequence
  Single strand DNA oligonucleotide
aaaggcaatttaccagtgatttct

SEQ ID NO: 52 - DNA, 19 bp, Artificial sequence
  Single strand DNA oligonucleotide
caaatggagaggtggcttg

SEQ ID NO: 53 - DNA, 22 bp, Artificial sequence
  Single strand DNA oligonucleotide
tcactcttcttccctgttctga

SEQ ID NO: 54 - DNA, 19 bp, Artificial sequence
  Single strand DNA oligonucleotide
acccatgtgcaggaactga

SEQ ID NO: 55 - DNA, 21 bp, Artificial sequence
  Single strand DNA oligonucleotide
ggaggtgattaaccagccaat

SEQ ID NO: 56 - DNA, 20 bp, Artificial sequence
  Single strand DNA oligonucleotide
aatgatgtcattgcccttgc

SEQ ID NO: 57 - DNA, 21 bp, Artificial sequence
  Single strand DNA oligonucleotide
gccaacagaccagacagtgtt

SEQ ID NO: 58 - DNA, 19 bp, Artificial sequence
  Single strand DNA oligonucleotide
tcttgcccttcctttcctg

SEQ ID NO: 59 - DNA, 19 bp, Artificial sequence
  Single strand DNA oligonucleotide
aagccagggacagatcagc

SEQ ID NO: 60 - DNA, 22 bp, Artificial sequence
  Single strand DNA oligonucleotide
ccacactctgtgcatttgaact

SEQ ID NO: 61 - DNA, 8 bp, Artificial sequence
  Single strand DNA oligonucleotide
uaacacug

SEQ ID NO: 62 - DNA, 8 bp, Artificial sequence
  Single strand DNA oligonucleotide
uaauacug

SEQ ID NO: 63 - DNA, 8 bp, Artificial sequence
  Single strand DNA oligonucleotide
uaauacug

SEQ ID NO: 64 - DNA, 8 bp, Artificial sequence
  Single strand DNA oligonucleotide
uggcucag

SEQ ID NO: 65 - DNA, 9 bp, Artificial sequence
  Single strand DNA oligonucleotide
tgctcgctt

SEQ ID NO: 66 - DNA, 9 bp, Artificial sequence
  Oligonucleotide sequences that can be used to form a loop
uucaagaga

SEQ ID NO: 67 - DNA, 9 bp, Artificial sequence
  Oligonucleotide sequences that can be used to form a loop
uuuguguag

SEQ ID NO: 68 - DNA, 58 bp, Artificial sequence
  siRNA capable of downregulating ZEB-1
ccggctgaacctcagacctagtaatctcgagattactaggtctgaggttcagtttttg

SEQ ID NO: 69 - DNA, 57 bp, Artificial sequence
  siRNA capable of downregulating ZEB-1
ccgggcaacaatacaagaggttaaactcgagtttaacctcttgtattgttgcttttt

SEQ ID NO: 70 - DNA, 57 bp, Artificial sequence
  siRNA capable of downregulating SOX-2
ccggcagctcgcagacctacatgaactcgagttcatgtaggtctgcgagctgttttt

SEQ ID NO: 71 - DNA, 57 bp, Artificial sequence
  siRNA capable of downregulating SOX-6
ccggccaacacttgtcagtaccattctcgagaatggtactgacaagtgttggttttt

SEQ ID NO: 72 - DNA, 68 bp, Artificial sequence
  miRNA
ccctcgtcttacccagcagtgtttgggtgcggttgggagtctctaatactgccgggtaatgatggagg

(19) United States
(12) Patent Application Publication
(10) Pub. No.: US 2016/0017290 A1
(43) Pub. Date: Jan. 21, 2016

(54) METHODS OF INCREASING INSULIN CONTENT IN CELLS

(71) Applicant: Ramot at Tel-Aviv University Ltd., Tel-Aviv (IL)

(72) Inventors: Shimon EFRAT, Zikhron-Yaakov (IL); Elad Sintov, Rehovot (IL)

(21) Appl. No.: 14/803,270

(22) Filed: Jul. 20, 2015

Related U.S. Application Data
(60) Provisional application No. 62/026,659, filed on Jul. 20, 2014.

(51) Int. Cl. C12N 5/071; A61K 35/39; C12N 15/113

(52) U.S. Cl. CPC C12N 5/0678; C12N 15/113; A61K 35/39; C12N 2310/141; C12N 2501/16; C12N 2501/335; C12N 2500/90

(57) ABSTRACT

A method of ex-vivo increasing insulin content in progenitor cells which express Zinc Finger E-Box Binding Homeobox 1 (ZEB-1) is disclosed. The method comprises contacting the progenitor cells with an inhibitory agent directed against a polypeptide, wherein the RNA transcript encoding said polypeptide is targeted by miRNA-200c, said polypeptide being selected from the group consisting of ZEB-1, SOX-2 and SOX-6.

### RELATED APPLICATION

This application claims the benefit of priority under 35 USC 119(e) of U.S. Provisional Patent Application No. 62/026,659 filed on Jul. 20, 2014, the contents of which are incorporated herein by reference in their entirety.

### SEQUENCE LISTING STATEMENT

The ASCII file, entitled 62684SequenceListing.txt, created on Jul. 20, 2015, comprising 15,218 bytes, submitted concurrently with the filing of this application is incorporated herein by reference.

### FIELD AND BACKGROUND OF THE INVENTION

The present invention, in some embodiments thereof, relates to methods of increasing insulin content in progenitor cells, and more particularly, but not exclusively, in dedifferentiated adult islet beta cells. The methods comprise use of agents which down-regulate ZEB-1, SOX-2 or SOX-6.

Type I diabetes is caused by the autoimmune destruction of the pancreatic islet insulin-producing beta cells. Insulin administration does not prevent the long-term complications of the disease, since the optimal insulin dosage is difficult to adjust.

Replacement of the damaged cells with regulated insulin-producing cells is considered the ultimate cure for type 1 diabetes. Pancreas transplantation has been successful but is severely limited by the shortage of donors. With the development of new islet isolation and immunosuppression procedures, significant success has been reported using islets from 2-3 donors per recipient (Shapiro A M, Lakey J R, Ryan E A et al. New Engl J Med 2000; 343:230-238). This progress underscores the urgent need for developing alternatives to human pancreas donors, namely abundant sources of cultured human β cells for transplantation.

Terminally differentiated, postmitotic islet cells are difficult to expand in tissue culture. Adult and fetal human islet cells grown on HTB-9 matrix in RPMI 1640 medium containing 11 mM glucose, and supplemented with 10% FBS and hepatocyte growth factor, were shown to proliferate at the most for 10-15 population doublings, after which they underwent senescence. The replication span could not be extended by expression of the catalytic subunit of human telomerase (hTERT), which was introduced into the cells with a retrovirus (Halvorsen T L, Beattie G M, Lopez A D, Hayek A, Levine F. J Endocrinol 2000; 166:103-109). Due to massive cell death, this method resulted in a 3-4 expansion of the islet cell mass.

The process of beta cell expansion by prolonged culture is accompanied by dedifferentiation of the cells.

In many instances, the dedifferentiation of cells is accompanied by drastic changes in phenotype in which their morphology changes from that of epithelial cells containing extensive cell-cell junctions and cytokeratin filament networks, to cells with a fibroblast or mesenchymal appearance. This process of dedifferentiation is known as an epithelial to mesenchymal transition (EMT).

A variety of signaling pathways, including Wnt/β-catenin, TGFβ-SMAD2/3, Hedgehog-GLI, receptor tyrosine kinases (RTKs), and Jagged1/Notch [6-8], have been implicated in upregulating the expression of several transcription factors important for EMT induction, including SNAIL (SNAI1), SLUG (SNAI2), TWIST, ZEB1 and ZEB2 (also called SIP1), all of which downregulate E-cadherin expression by repression of CDH1. ZEB1 and ZEB2, members of the zinc-finger homeobox (ZFH) family of repressors, activate EMT by binding to E-box elements present in the E-cadherin promoter and suppressing its activity. ZEB1 also promotes EMT by repressing expression of basement membrane components and cell polarity proteins.

U.S. Patent Application Publication No. 2006/0292127 teaches dedifferentiating, and not redifferentiating, beta cells by contacting the cells with agents that regulate Snail/slug/slit family of transcription factors.

International Application No. WO2006/054305 teaches redifferentiation of expanded pancreatic beta cells in a medium comprising betacellulin and/or ngn-3.

U.S. Pat. No. 8,728,813 teaches downregulation of SLUG during redifferentiation of expanded pancreatic beta cells.

Additional background art includes Shirakihara, T., et al. Mol Biol Cell, 2007. 18(9): p. 3533-44; Eger, A. et al., Oncogene, 2005. 24(14): p. 2375-85; and Aigner K, et al. Oncogene 2007; 26 (49):6979-88.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B. Induction of ZEB expression during islet cell dedifferentiation. A, qPCR analysis of RNA extracted from expanded islet cells at the indicated passages. Values are mean±SE, relative to uncultured islets (n=3-6 donors), and normalized to GAPDH, or RPLPO and TBP. *p<0.05. B, Immunoblotting analysis of protein extracted from expanded islet cells at the indicated passages. HSC70, heat-shock cognate 70. Values are mean±SE, relative to uncultured islets (n=3-7 donors). *p<0.05, **p<0.01.

FIGS. 2A-D. ZEB1 inhibition restores insulin expression in expanded islet cells and blocks BCD cell replication. A, ZEB1 inhibition by shRNA Immunoblotting analysis of expanded islet cells infected at passage 6 with lentiviruses expressing ZEB1 (shRNA#1, TRCN-17563; shRNA#2, TRCN-17566) or control shRNA and analyzed 7 days later. Percent inhibition is mean±SE (n=3 donors; p=1×10⁻⁵ for shRNA#1, p=0.004 for shRNA#2). B, qPCR analysis of expanded islet cells infected at passages 4-6 with increasing amounts of ZEB1 shRNA#1 or control shRNA lentiviruses. Values are mean±SE (n=3-5 donors), normalized to human RPLPO and TBP, relative to cells infected with control shRNA at MOI of 3 (RQ=1). *p<0.05, **p<0.01, relative to cells infected with control shRNA. C, qPCR analysis of expanded islet cells infected at passages 4-6 with ZEB1 or control shRNA lentiviruses at MOI 3:1. UI, uninfected cells. Values are mean±SE (n=3-5 donors) and normalized to human RPLPO and TBP. D, Immunofluorescence analysis of expanded islet cells, labeled with eGFP and infected at passages 6-7 with lentiviruses expressing ZEB1 or control shRNAs, using antibodies for Ki67 and eGFP. Values are mean±SE (n=3 donors), based on counting >200 cells for each donor.

FIGS. 3A-D. Redifferentiation of BCD cells induced by ZEB1 inhibition. Expanded islet cells transduced with the lineage tracing lentiviruses were sorted by FACS at passages 2-3. eGFP-labeled BCD cells were then grown until passages 6-7 and transduced with lentiviruses expressing ZEB1 or control shRNAs. RNA was extracted 7 days later. A, qPCR analysis. B, C, cDNA microarray analysis (n=4 donors). B, Hierarchical cluster analysis. C, DAVID functional annotation. D, qPCR validation of genes listed in Table 4. A, D, Values are mean±SE (n=3-4 donors) relative to UI and normalized to human RPLPO and TBP. UI, uninfected cells.

FIG. 4. ZEB1 inhibition induces human C-peptide release from expanded islet cells transplanted into NSG mice. 1-2×10⁶ expanded islet cells infected at passages 5-6 with lentiviruses expressing ZEB1 or control shRNAs were transplanted 7 days later under the renal capsule of >12-w male NSG mice. Serum samples were collected 42-57 days later and assayed for human C-peptide by ELISA. Values are mean±SE (n=4 mice transplanted with ZEB1 shRNA-treated cells, and 3 mice transplanted with control shRNA-treated cells, from 3 different donors).

FIGS. 5A-G. Synergistic effect of ZEB1 inhibition and RC treatment. A,B, qPCR analysis of transcripts encoding β-cell proteins (A) and cell adhesion molecules (B) in RNA extracted from expanded islet cells 7 days following infection at passages 5-7 with lentiviruses expressing ZEB1 or control shRNAs, and an 8-day treatment with RC. Values are mean±SE (n=3-5 donors) relative to untreated cells (UTR; RQ=1) and normalized to human RPLPO and TBP. UI, uninfected cells treated with RC. C, Immuonflourescense analysis of expanded islet cells, 7 days following infection at passages 5-7 with lentiviruses expressing ZEB1 or control shRNAs, and a 4-day treatment with RC. Percent of double-positive cells are mean±SE (n=3 donors), based on counting >500 cells from each donor. Cells treated with ZEB1 shRNA are displayed. Bar=25 μm. D, Human C-peptide content of expanded islet cells 7 days following infection at passages 6-7 with lentiviruses expressing ZEB1 or control shRNAs, and a 4-day treatment with RC. Values are mean±SE (n=3 donors). E, Glucose-induced insulin secretion from expanded islet cells 7 days following infection at passages 6-7 with lentiviruses expressing ZEB1 or control shRNAs, following a 4-day RC treatment. Values are mean±SE (n=3-4 donors) of human C-peptide released in the presence of 16.7 mM glucose, relative to 0 mM glucose. F, qPCR analysis of BCD cells sorted at passages 2-3, infected at passages 6-7 with lentiviruses expressing ZEB1 or control shRNAs, and treated 7 days later with RC for 4 days. Values are mean±SE (n=3-4 donors) relative to UTR (RQ=1) and normalized to human RPLPO and TBP. G, Immuonflourescence analysis of eGFP-labeled islet cells infected at passage 5-7 with ZEB1 or control shRNA lentiviruses and treated 7 days later with RC for 4 days. Values are mean±SE (n=3 donors), based on counting >300 cells in all donors. Bar=50 μm.

FIGS. 6A-H. Insulin expression and cell proliferation in expanded islet cells is regulated by the ZEB1/miR-200 negative feedback loop. A, qPCR analysis of miR-200c expression in FACS-sorted BCD cells infected at passages 6-7 with lentiviruses expressing ZEB1 or control shRNAs. UI, uninfected cells. Values are mean±SE (n=3 donors), relative to UI, and normalized to human U6 snRNA and miR-24. B, qPCR analysis of changes in miR-200c levels in expanded islet cells treated at passages 5-7 with RC for the indicated times. Values are mean±SE (n=3 donors), relative to UTR, and normalized to human U6 snRNA and miR-24. C,D, Immunoblotting analysis of protein extracted from expanded islet cells infected at passages 3-4 with retroviruses expressing human miR-200c or an empty vector (C), or at passages 5-6 with lentiviruses expressing ZEB1 or control shRNAs (D). HSC70, heat-shock cognate 70. Values are mean±SE (n=4 donors). E, Effect of SOX6 shRNA on insulin expression. qPCR analysis of expanded islet cells infected at passages 5-6 with lentiviruses expressing SOX6 shRNA#1 (TRCN-017988), shRNA#2 (TRCN-017991), or control shRNA. Values are mean±SE (n=3-4 donors), relative to UI, and normalized to human RPLPO. Indicated fold change and p-values are relative to control shRNA. *p<0.05, **p<0.01, **p<0.005. F, qPCR analysis of FACS-sorted BCD cells infected at passages 6-7 with lentiviruses expressing ZEB1 or control shRNAs. Values are mean±SE (n=3 donors), relative to UI, and normalized to human RPLPO and TBP. G, Effect of SOX2 shRNAs on expression of cell cycle regulators. qPCR analysis of expanded islet cells infected at passage 6 with lentiviruses expressing SOX2 shRNA#1 (TRCN-010772), shRNA#2 (TRCN-355694), or control shRNA. Values are mean±SE (n=3-4 donors), relative to UI, and normalized to human RPLPO. Indicated fold change and p-values are relative to control shRNA. *p<0.05, **p<0.01. SOX2 shRNAs inhibited SOX2 transcript levels by up to 28±22% (n=4 donors, p=0.06). H. A proposed model for mediation of ZEB1 effects on PDX1-induced insulin gene expression and cell cycle through miR-200c, SOX6, and SOX2.

### SUMMARY OF THE INVENTION

According to an aspect of some embodiments of the present invention there is provided a method of ex-vivo increasing insulin content in progenitor cells which express Zinc Finger E-Box Binding Homeobox 1 (ZEB-1), comprising contacting the progenitor cells with an inhibitory agent directed against a polypeptide, wherein the RNA transcript encoding the polypeptide is targeted by miRNA-200c, the polypeptide being selected from the group consisting of ZEB-1, SOX-2 and SOX-6, thereby increasing insulin content in the progenitor cells.

According to an aspect of some embodiments of the present invention there is provided an isolated population of cells, comprising a heterologous polynucleotide which down-regulates a polypeptide, wherein the RNA transcript encoding the polypeptide is targeted by miRNA-200c, the polypeptide being selected from the group consisting of ZEB-1, SOX-2 and SOX-6, wherein the cells secrete insulin.

According to an aspect of some embodiments of the present invention there is provided an isolated population of cells generated according to the method described herein.

According to an aspect of some embodiments of the present invention there is provided a method of treating diabetes in a subject, comprising transplanting a therapeutically effective amount of the population of adult islet beta cells described herein into the subject, thereby treating diabetes.

According to an aspect of some embodiments of the present invention there is provided a pharmaceutical composition comprising as an active ingredient the population of cells described herein and a pharmaceutically acceptable carrier.

According to an aspect of some embodiments of the present invention there is provided a method of treating Diabetes in a subject in need thereof comprising administering to the subject a therapeutically effective amount of an inhibitory agent directed against a polypeptide, wherein the RNA transcript encoding the polypeptide is targeted by miRNA-200c, the polypeptide being selected from the group consisting of ZEB-1, SOX-2 and SOX-6, thereby treating the Diabetes.

According to some embodiments of the invention, the progenitor cells are selected from the group consisting of dedifferentiated adult islet beta cells, mesenchymal stem cells and induced pluripotent stem cells dedifferentiated from beta cells.

According to some embodiments of the invention, the inhibitory agent is a polynucleotide agent.

According to some embodiments of the invention, the method further comprises culturing the progenitor cells in a medium comprising nicotinamide, exendin-4, activin A and glucose, the culture medium being devoid of serum.

According to some embodiments of the invention, culturing is effected following or concomitant with the contacting.

According to some embodiments of the invention, the method comprises:

(a) exposing the progenitor cells to a culture medium comprising the nicotinamide, the exendin-4, the activin A, wherein the glucose is present at a concentration of 10-100 mM; and subsequently

(b) exposing the progenitor cells in an additional culture medium comprising the nicotinamide and the exendin-4, wherein the glucose is present at a concentration of 0.5-10 mM.

According to some embodiments of the invention, additional medium is devoid of activin A.

According to some embodiments of the invention, the method further comprises contacting the progenitor cells with an agent that down-regulates an activity and/or amount of HES-1.

According to some embodiments of the invention, the dedifferentiated adult islet beta cells comprise induced pluripotent stem cells generated from beta cells.

According to some embodiments of the invention, the dedifferentiated adult islet beta cells are generated by culturing the adult islet beta cells for at least 10 passages.

According to some embodiments of the invention, the culturing is effected in CMRL medium.

According to some embodiments of the invention, the isolated population of cells is genetically modified to express a pharmaceutical agent.

According to some embodiments of the invention, the isolated populations of cells are adult islet beta cells.

According to some embodiments of the invention, the inhibitory agent is a polynucleotide agent.

According to some embodiments of the invention, the inhibitory agent is a small molecule agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to methods of increasing insulin content in progenitor cells which express Zinc Finger E-Box Binding Homeobox 1 (ZEB-1), and more particularly, but not exclusively, in dedifferentiated adult islet beta cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In-vitro expansion of functional adult human β-cells is an attractive approach for generating insulin-producing cells for transplantation. However, human islet cell expansion in culture results in loss of β-cell phenotype and epithelial-mesenchymal transition (EMT).

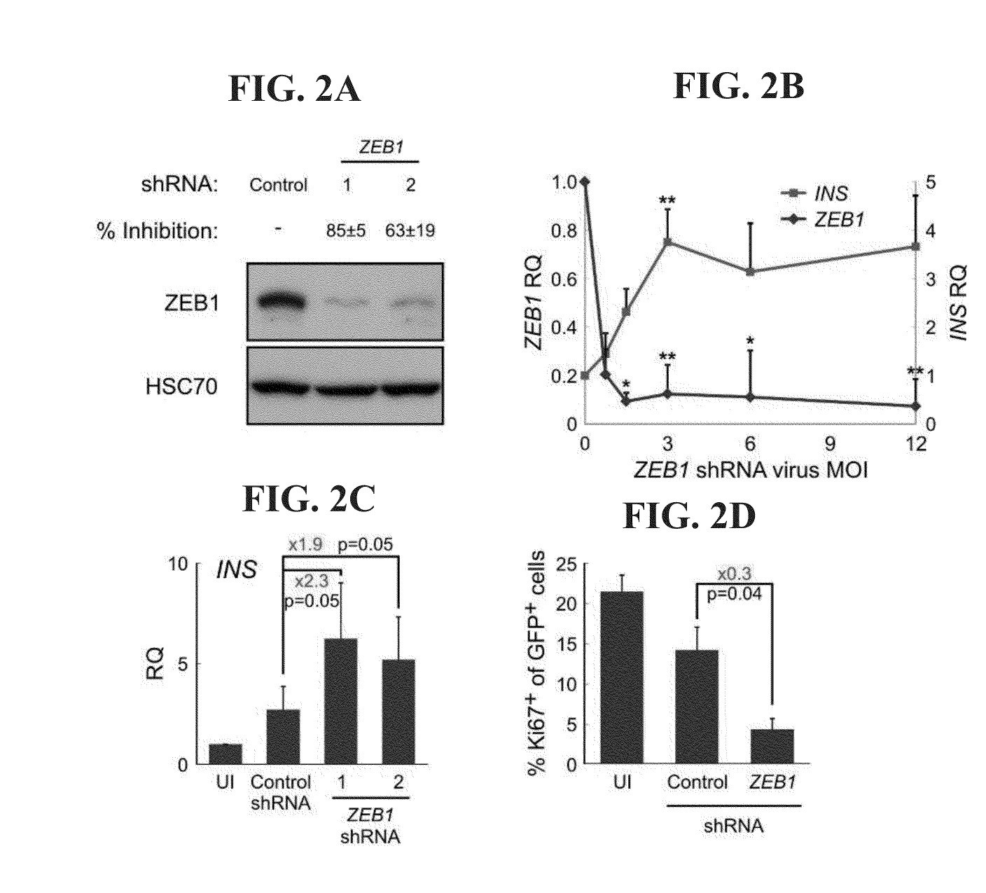

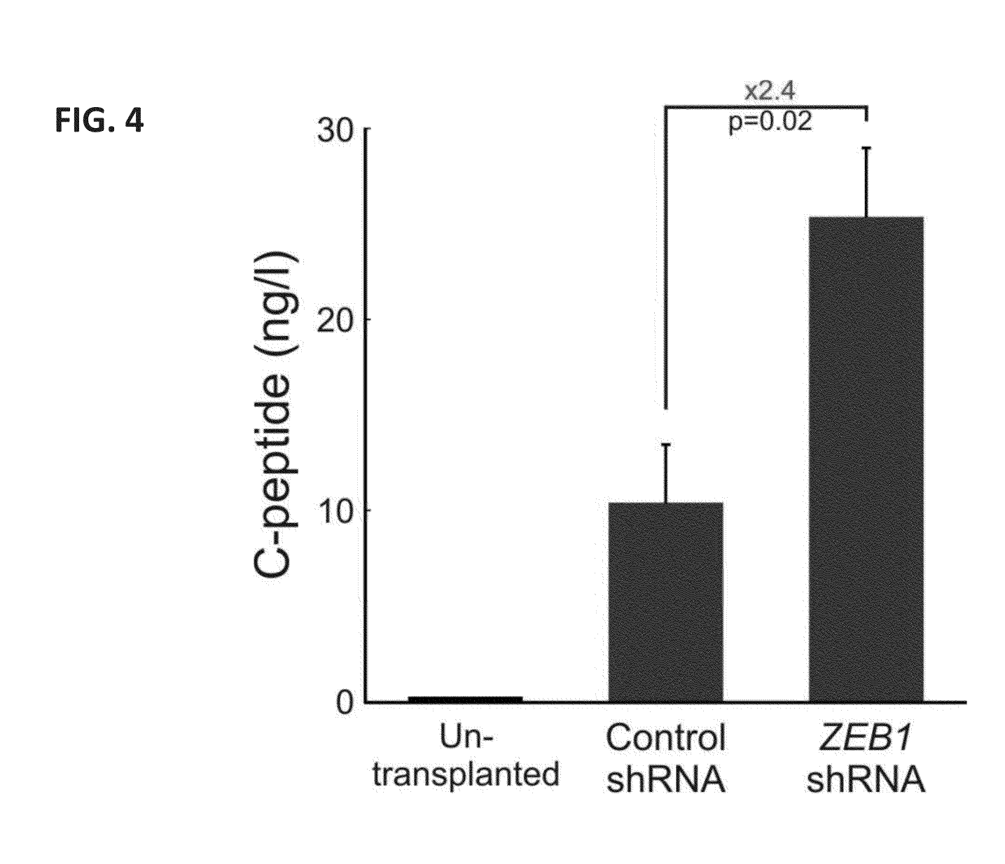

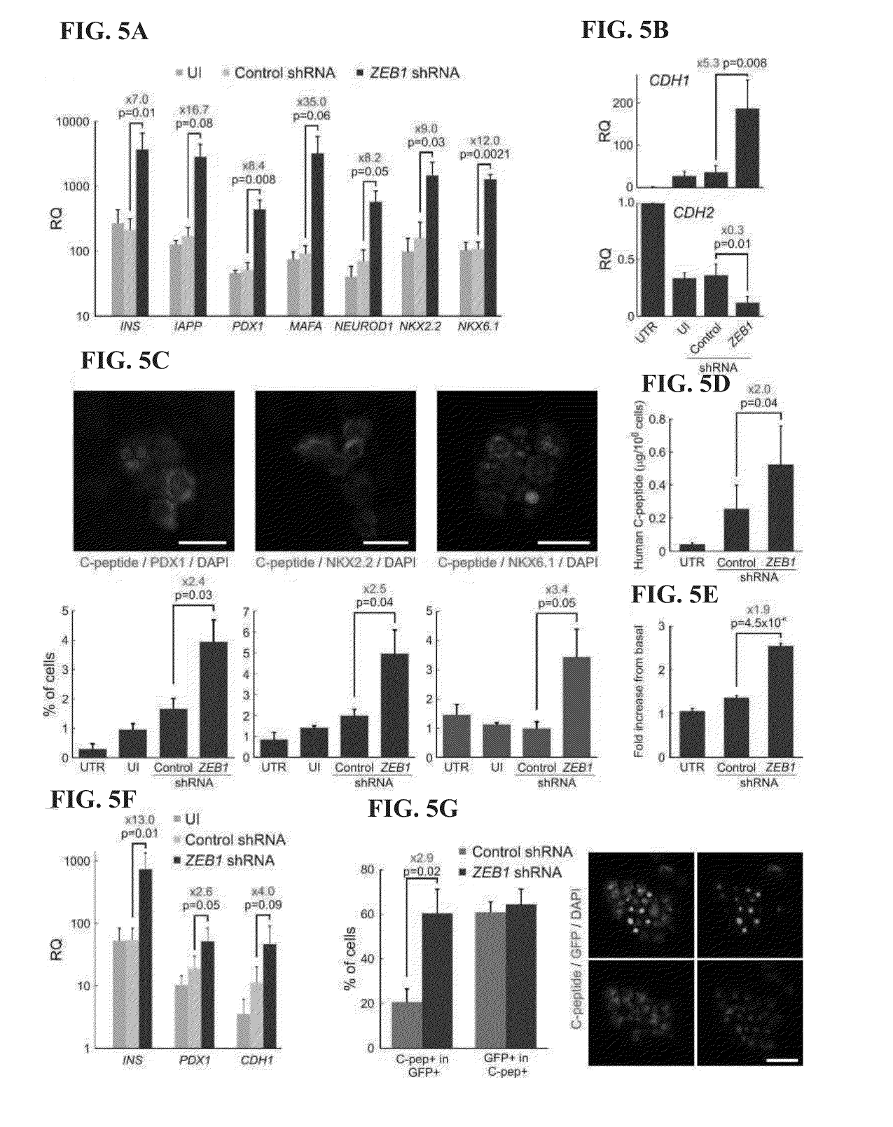

The present inventors have now shown that the process of dedifferentiation activates expression of ZEB1 and ZEB2, two members of the zinc-finger homeobox family of E-cadherin repressors, which play key roles in EMT. Downregulation of ZEB1 using shRNA in expanded β-cell-derived (BCD) cells induced mesenchymal-epithelial transition (MET), β-cell gene expression, and proliferation attenuation (FIGS. 2A-D). In addition, inhibition of ZEB1 expression potentiated redifferentiation induced by a combination of soluble factors, as judged by an increased number of C-peptide-positive cells and an improved response to glucose stimulation (FIGS. 5A-G). Furthermore, ZEB1 shRNA led to increased insulin secretion in cells transplanted in vivo (FIG. 4).

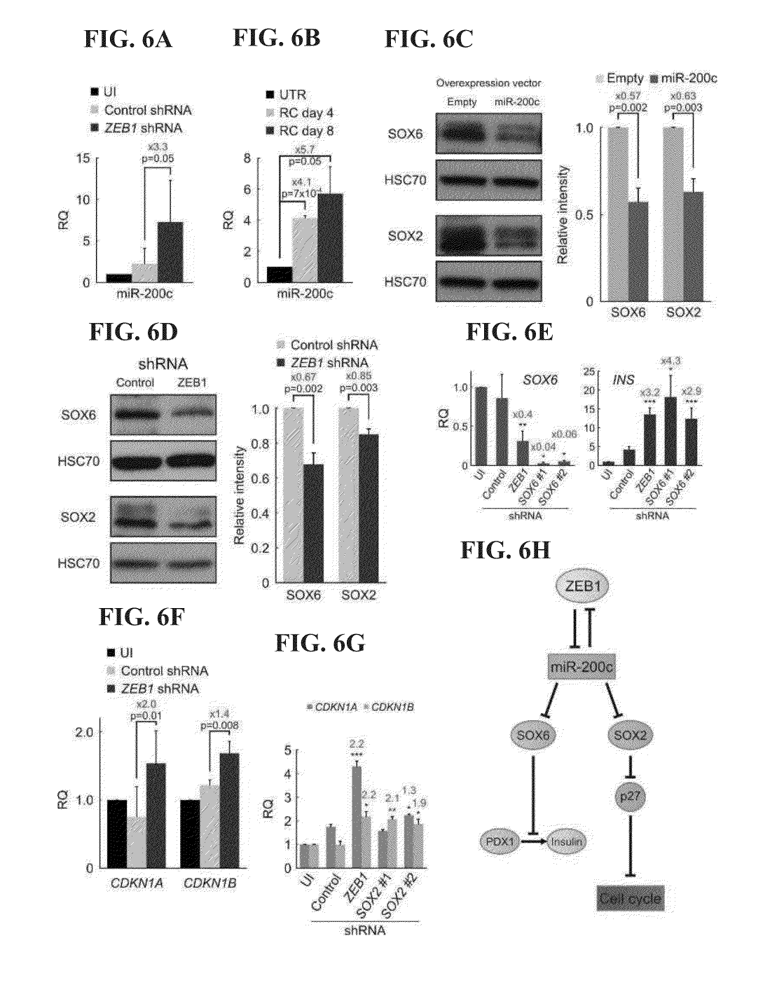

Whilst further reducing the present invention to practice, the present inventors found that down-regulation of the miR-200c target gene SOX6 also brought about an increase in insulin expression, as measured by RT-PCR (FIG. 6E). These findings support the proposal that the effects of ZEB1 inhibition are mediated by attenuation of the miR-200c target genes SOX6 and SOX2 (FIG. 6H). In addition, the results emphasize the key role of ZEB1 in EMT in cultured BCD cells and support the value of ZEB1 inhibition for BCD cell redifferentiation and generation of functional human β-like cells for cell therapy of diabetes.

Thus, according to a first aspect of the present invention there is provided a method of ex-vivo increasing insulin content in progenitor cells which express Zinc Finger E-Box Binding Homeobox 1 (ZEB-1), comprising contacting the progenitor cells with an inhibitory agent directed against a polypeptide, wherein the RNA transcript encoding the polypeptide is targeted by miRNA-200c, the polypeptide being selected from the group consisting of ZEB-1, SOX-2 and SOX-6, thereby increasing insulin content in the progenitor cells.

As used herein the phrase “ex-vivo” refers to cells which are removed from a living organism and cultured outside the organism (e.g., in a test tube, or on a culture disc).

As used herein, the term “progenitor cell” refers to a cell that has the capacity to differentiate into a more “mature” cell. According to a particular embodiment, the progenitor cell is within the same cell-type/lineage as a pancreatic beta cell.

Examples of progenitor cells contemplated by the present invention include but are not limited to dedifferentiated adult islet beta cells, mesenchymal stem cells and induced pluripotent stem cells dedifferentiated from beta cells, each of which are described herein under.

According to a particular embodiment, the progenitor cells are human cells.

Dedifferentiated Adult Islet Beta Cells

As used herein, the phrase “adult islet beta cells” refers to post-natal (e.g., non-embryonic) pancreatic islet endocrine cells which are capable of secreting insulin in response to elevated glucose concentrations and express typical beta cell markers. Examples of beta cell markers include, but are not limited to, insulin and pdx.

The isolated adult islet beta cells of this aspect of the present invention may be of homogeneous or heterogeneous nature.

Thus, for example, the adult islet beta cells of this aspect of the present invention may be comprised in isolated pancreatic islets. Islet cells are comprised of the following: 1) beta cells that produce insulin; 2) alpha cells that produce glucagon; 3) delta cells (or D cells) that produce somatostatin; and/or F cells that produce pancreatic polypeptide. The polypeptide hormones (insulin, glucagon, somatostatin and pancreatic polypeptide) inside these cells are stored in secretary vesicles in the form of secretory granules.

Methods of isolating islets are well known in the art. For example, islets may be isolated from pancreatic tissue using collagenase and ficoll gradients. Preferably the adult islet beta cells of the present invention are dispersed into a single cell suspension—e.g. by the addition of trypsin or by trituration.

The adult islet beta cells may be further isolated being substantially free from other substances (e.g., other cells, proteins, nucleic acids, etc.) that are present in its in-vivo environment e.g. by FACs sorting.

The adult islet beta cells may be obtained from any autologous or non-autologous (i.e., allogeneic or xenogeneic) mammalian donor. For example, cells may be isolated from a human cadaver.

Dedifferentiation of the adult islet beta cells may be effected by expansion (i.e. culturing) for a prolonged number of passages—e.g. in CMRL medium.

As used herein, the term “CMRL 1066” refers to the serum free medium, originally developed by Connaught Medical Research Laboratories for the culture of L cells, and includes any other derivations thereof provided that the basic function of CMRL is preserved. CMRL-1060 medium is commercially available in either liquid or powder form from companies including Gibco BRL, Grand Island, N.Y., catalogue number 11530-037; Cell and Molecular Technologies, Phillipsburg N.J.; Biofluids Inc, Rockville, Md.; Bioreclamation Inc. East Meadow, N.Y.; United States Biological, Swampscott, Mass.; Sigma Chemical Company, St. Louis, Mo.; Cellgro/Mediatech, Herndon, Va. and Life technologies, Rockville Md.

The medium used to culture the beta cells may further comprise supplementary constituents which may improve growth and/or viability thereof. These include, but are not limited to, growth factors (e.g. hepatocyte growth factor, nerve growth factor and/or epidermal growth factor) serum (e.g. fetal calf serum or fetal bovine serum), glucose (e.g. 5.6 mM) and antibiotics.

Non-apoptotic culturing conditions for adult islet beta cells are known in the art—see for example U.S. Patent Application Publication No. 20080014182. According to one embodiment, beta cells are passaged every seven days and refed twice a week. According to the teachings of U.S. Patent Application No. 20080014182, adult islet beta cells may be expanded 65,000 fold without any detectable apoptosis. According to another embodiment, the adult islet beta cells are propagated as anchorage-dependent cells by attaching to a solid substrate (i.e., a monolayer type of cell growth).

As used herein, the term “expanding” refers to increasing the number and overall mass of adult islet beta cells of the present invention by the process of cell division, rather than simply enlarging by hypertrophy.

According to one embodiment, the cells are expanded in Mesencult XF medium comprising glucose at a concentration of about 10-100 mM glucose, more preferably 10-50 mM, more preferably 10-25 mM, such as for example 25 mM.

The cells are preferably expanded for at least 6 passages, 7 passages, 8 passages, 9 passages, 10 passages, 11 passages, 12 passages, 13 passages, 14 passages, 15 passages or at least 16 passages.

According to a particular embodiment, the expanding is effected under adherent conditions which comprise incubating on an attachment medium selected from the group consisting of laminin, fibronectin, matrigel and Mesencult XF attachment substrate.

Mesenchymal Stem Cells:

Mesenchymal stem cells may be isolated from various tissues including but not limited to bone marrow, peripheral blood, blood, placenta and adipose tissue. A method of isolating mesenchymal stem cells from peripheral blood is described by Kassis et al. [Bone Marrow Transplant. 2006 May; 37(10):967-76]. A method of isolating mesenchymal stem cells from placental tissue is described by Brooke G et al. [Br J Haematol. 2009 February; 144 (4):571-9].

Methods of isolating and culturing adipose tissue, placental and cord blood mesenchymal stem cells are described by Kern et al. [Stem Cells, 2006; 24:1294-1301].

According to a preferred embodiment of this aspect of the present invention, the mesenchymal stem cells are human.

Bone marrow can be isolated from the iliac crest or the sternum of an individual by aspiration. Low-density BM mononuclear cells (BMMNC) may be separated by FICOLL-PAQUE density gradient centrifugation. In order to obtain mesenchymal stem cells, a cell population comprising the mesenchymal stem cells (e.g. BMMNC) may be cultured in a proliferating medium capable of maintaining and/or expanding the cells in the presence of platelet lysate. According to one embodiment the populations are plated on plastic surfaces (e.g. in a flask) and mesenchymal stem cells are isolated by removing non-adherent cells. Alternatively mesenchymal stem cell may be isolated by FACS using mesenchymal stem cell markers including for example nucleostemin, an intracellular protein which is expressed in MSCs but not differentiated cells. In addition, cell surface markers for undifferentiated MSCs include receptors for Bone Morphogenetic Proteins (BMPR), Endoglin, Stem Cell Factor Receptor (SCF R), and STRO-1.

Induced Pluripotent Stem Cells Dedifferentiated from Beta Cells:

Dedifferentiation of adult islet beta cells may be effected by transfecting the cells with genes known to generate induced pluripotent stem cells (iPS) cells.

According to one embodiment the method is effected by expressing in the cells at least one polypeptide belonging to the Oct family or the Sox family.

According to another embodiment, the method is effected by expressing in the cells at least two polypeptides—one belonging to the Oct family and one to the Sox family.

Examples of polypeptides belonging to the Oct family include, for example, Oct3/4 (NM_{—}013633, mouse and NM_{—}002701, human), Oct1A (NM_{—}198934, mouse and NM_{—}002697, human), Oct6 (NM_{—}011141, mouse and NM_{—}002699, human), and the like. Oct3/4 is a transcription factor belonging to the POU family, and is reported as a marker of undifferentiated cells (Okamoto et al., Cell 60:461-72, 1990). Oct3/4 is also reported to participate in the maintenance of pluripotency (Nichols et al., Cell 95:379-91, 1998).

Examples of polypeptides belonging to the Sox (SRY-box containing) family include, for example Sox1 (NM_{—}009233, mouse and NM_{—}005986, human), Sox3 (NM_{—}009237, mouse and NM_{—}005634, human), Sox7 (NM_{—}011446, mouse and NM_{—}031439, human), Sox15 (NM_{—}009235, mouse and NM_{—}006942, human), Sox17 (NM_{—}011441, mouse and NM_{—}022454, human) and Sox18 (NM_{—}009236, mouse and NM_{—}018419, human), and a preferred example includes Sox2 (NM_{—}011443, mouse and NM_{—}003106, human).

According to yet another embodiment, the method is effected by expressing in the cells four polypeptides—one belonging to the Oct family, one belonging to the Sox family, Nanog and lin28.

Alternatively, the method is effected by expressing in the cells four polypeptides—one belonging to the Oct family, one belonging to the Sox family, Klf-4 and c-Myc.

Expressing the dedifferentiating factors described herein above in the expanded pancreatic beta cells may be performed by genetic manipulation—example using expression constructs. Various methods can be used to introduce the expression vectors of the present invention into the pancreatic beta cells. Such methods are generally described in, for instance: Sambrook, J. and Russell, D. W. (1989, 1992, 2001), Molecular Cloning: A Laboratory Manual, Cold Springs Harbor Laboratory, New York; Ausubel, R. M. et al., eds. (1994, 1989). Current Protocols in Molecular Biology, John Wiley and Sons, Baltimore, Md. (1989); Chang, P. L., ed. (1995). Somatic Gene Therapy, CRC Press, Boca Raton, Fla.; Vega, M. A. (1995). Gene Targeting, CRC Press, Boca Raton, Fla.; Rodriguez, R. L. and Denhardt, D. H. (1987). Vectors: A Survey of Molecular Cloning Vectors and Their Uses, Butterworth-Heinemann, Boston, Mass.; and Gilboa, E. et al. (1986). Transfer and expression of cloned genes using retro-viral vectors. Biotechniques 4(6), 504-512; and include, for example, stable or transient transfection, lipofection, electroporation, and infection with recombinant viral vectors. In addition, see U.S. Pat. Nos. 5,464,764 and 5,487,992 for positive-negative selection methods.

Introduction of the expression constructs of the present invention into the pancreatic beta cells by viral infection offers several advantages over other methods such as lipofection and electroporation offering higher efficiency of transformation and propagation. According to a particular embodiment, expressing the dedifferentiating factors described herein above in the pancreatic beta cells is performed by retroviral transduction.

Methods of inducing iPS cells without viral integration are also contemplated—see for example Stadtfeld et al., 2008, [Science 322, 945-949] and Okita et al., 2008, [Science 322, 949-953].

Alternatively, the expanded pancreatic beta cells may be transfected with mRNAs encoding the dedifferentiating factors [Givol et al., BBRC 394(2010): 189-193; Warren et al., Cell Stem Cell, Volume 7, Issue 5, 5 Nov. 2010, Pages 549-550] or by introduction of the proteins themselves (see for example Kim, D. et al. Cell Stem Cell doi:10.1016/j.stem.2009.05.005 (2009) and Zhou, H. Et al. Cell Stem Cell 4, 381-384, (2009).

As mentioned, the methods of this aspect of the present invention are used to increase insulin content in the above mentioned cells.

Methods of measuring insulin content are known in the art and include the use of ELISA, immunohistochemistry or radioimmunoassay.

Redifferentiation of the cells is effected using an inhibitor which is directed against Zinc Finger E-Box Binding Homeobox 1 (ZEB-1), SOX-2 and/or SOX-6.

As used herein the term “re-differentiating” refers to the altering of a cell such that it passes from one of a less defined function to one of a more defined function (may also be referred to as more differentiated). For example, the defined functions of an adult beta cell include storing insulin and secreting insulin in response to glucose. Re-differentiation of the expanded adult islet beta cells of the present invention may include such processes as increasing beta cell insulin content, increasing sensitivity to glucose and/or increasing secretory apparatus. Methods of increasing beta cell insulin content may include increasing insulin transcription and/or post transcriptional control and/or increasing translation and/or post-translational control. Methods of increasing beta cell insulin content may also include enhancing insulin storage and/or retarding insulin breakdown. Methods of increasing sensitivity to glucose may include increasing the expression of glucose transporters.

According to a particular embodiment, redifferentiation of the cells is effected using an inhibitor of ZEB-1 or SOX-6.

Zinc Finger E-Box Binding Homeobox 1 (ZEB-1) is a zinc finger transcription factor (UniProt No. P37245).

The mRNAs encoding this factor is set forth in NM_{—}030751. Alternative transcripts are set forth in NM_{—}001128128.2, NM_{—}001174093.1, NM_{—}001174094.1, NM_{—}001174095.1, NM_{—}001174096.1 and NM_{—}030751.5.

The protein sequence of ZEB-1 is set forth in NP_{—}110378. Alternative transcripts are set forth in NP_{—}001121600.1, NP_{—}001167564.1, NP_{—}001167565.1, NP_{—}001167566.1, NP_{—}001167567.1 and NP_{—}110378.3.

SOX-6 (Swiss Prot No. P35712) is a transcription factor characterized by a conserved DNA-binding domain and its ability to bind the minor groove of DNA. The encoded protein is a transcriptional activator that is required for normal development of the central nervous system, chondrogenesis and maintenance of cardiac and skeletal muscle cells.

mRNAs encoding this factor are set forth in NM_{—}001145811.1, NM_{—}001145819.1, NM_{—}017508.2 and NM_{—}033326.3.

SOX-2 (NP_{—}003097) is a member of the Sox family of transcription factors, which have been shown to play key roles in many stages of mammalian development.

This protein family shares highly conserved DNA binding domains known as HMG (High-mobility group) box domains containing approximately 80 amino acids.

A mRNA encoding this factor is set forth in NM_{—}003106.

Following is a list of agents capable of downregulating expression level and/or activity of ZEB-1, SOX-2 or SOX-6.

One example, of an agent capable of downregulating ZEB-1, SOX-2 or SOX-6 is an antibody or antibody fragment capable of specifically binding thereto. Preferably, the antibody specifically binds at least one epitope of ZEB-1, SOX-2 or SOX-6. As used herein, the term “epitope” refers to any antigenic determinant on an antigen to which the paratope of an antibody binds.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Downregulation of ZEB-1, SOX-2 or SOX-6 can be also achieved by RNA silencing.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated.

Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., ZEB-1, SOX-2 or SOX-6) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi: 10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11.

Some of the nucleotides in the loop can be involved in base-pair interactions with other nucleotides in the loop. Examples of oligonucleotide sequences that can be used to form the loop include 5′-UUCAAGAGA-3′ (SEQ ID NO: 66; Brummelkamp, T. R. et al. (2002) Science 296: 550) and 5′-UUUGUGUAG-3′ (SEQ ID NO: 67; Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Examples of suitable siRNA capable of downregulating ZEB-1 can be the following sequences:

(SEQ ID NO: 68)
CCGGCTGAACCTCAGACCTAGTAATCTCGAGATTACTAGGTCTGAG
GTTCAGTTTTTG
or
(SEQ ID NO: 69)
CCGGGCAACAATACAAGAGGTTAAACTCGAGTTTAACCTCTTGTAT
TGTTGCTTTTT.
An example of a suitable siRNA capable of
downregulating SOX-2 can be the
following sequence:
(SEQ ID NO: 70)
CCGGCAGCTCGCAGACCTACATGAACTCGAGTTCATGTAGGTCTGC
GAGCTGTTTTT.
An example of a suitable siRNA capable of
downregulating SOX-6 can be the
following sequence:
SEQ ID NO: 71
CCGGCCAACACTTGTCAGTACCATTCTCGAGAATGGTACTGACAAG
TGTTGGTTTTT.

According to another embodiment the RNA silencing agent may be a miRNA or miRNA mimic.

miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

An exemplary miRNA capable of downregulating ZEB-1, SOX-2 and SOX-6 is miR-200.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence:

(SEQ ID NO: 72)
CCCTCGTCTTACCCAGCAGTGTTTGGGTGCGGTTGGGAGTCTCTAATACT
GCCGGGTAATGATGGAGG
or
variants thereof.

Preparation of miRNAs mimics can be effected by any method known in the art such as chemical synthesis or recombinant methods.

Synthesis of RNA silencing agents suitable for use with some embodiments of the invention can be effected as follows. First, the selected mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of some embodiments of the invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

Another agent capable of downregulating ZEB-1/SOX-2/SOX-6 is a DNAzyme molecule capable of specifically cleaving an mRNA transcript or DNA sequence of the ZEB-1/SOX-2/SOX-6.

DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002))].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www(dot)asgt(dot)org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of ZEB-1/SOX-2/SOX-6 can also be effected by using an antisense polynucleotide capable of specifically hybridizing with an mRNA transcript encoding ZEB-1/SOX-2/SOX-6.

Another agent capable of downregulating ZEB-1/SOX-2/SOX-6 is a ribozyme molecule capable of specifically cleaving an mRNA transcript encoding ZEB-1/SOX-2/SOX-6. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of regulating the expression of ZEB-1/SOX-2/SOX-6genes in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al., Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the ZEB-1/SOX-2/SOX-6 regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al., J Biol Chem, 2001; 276:28991-98), and the sequence- and target specific downregulation of expression of the Ets2 transcription factor, important in prostate cancer etiology (Carbone, et al., Nucl Acid Res. 2003; 31:833-43), and the pro-inflammatory ICAM-1 gene (Besch et al., J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Publication Nos. 2003/017068 and 2003/0096980 to Froehler et al., and 2002/0128218 and 2002/0123476 to Emanuele et al., and U.S. Pat. No. 5,721,138 to Lawn.

Another agent capable of downregulating ZEB-1 would be any molecule which binds to and/or cleaves ZEB-1/SOX-2/SOX-6. Such molecules can be antagonists, or inhibitory peptides.

It will be appreciated that a non-functional analogue of at least a catalytic or binding portion of ZEB-1/SOX-2/SOX-6 can be also used as an agent which downregulates the proteins.

Another agent which can be used along with some embodiments of the invention to downregulate ZEB-1/SOX-2/SOX-6 is a molecule which prevents ZEB-1/SOX-2/SOX-6 activation or DNA binding.

Together with down-regulation of ZEB-1/SOX-2/SOX-6, the present invention also contemplates culturing the cells in a medium comprising differentiation factors under conditions to allow differentiation of the cells into insulin producing cells.

Exemplary mediums have been disclosed in U.S. Pat. No. 8,728,813, U.S. Patent Application Publication Nos. 20080014182, 20120003739, each of which are incorporated herein by reference.

According to one embodiment, the cells are redifferentiated in a culture medium comprising nicotinamide, exendin-4, activin A and 10-50 mM glucose, the culture medium being devoid of serum.

Exemplary culture medium contemplated by the present inventors which may be used to redifferentiate the progenitor cells include CMRL-1066, DMEM, RPMI etc.

Exemplary concentration ranges of nicotinamide include 1-100 mM, more preferably 1-50 mM, more preferably 1-20 mM, such as for example 10 mM.

Exemplary concentration ranges of exendin 4 include 1-100 nM, more preferably 1-50 nM, more preferably 1-20 nM, such as for example 8 nM.

Exemplary concentration ranges of activin A include 1-100 nM, more preferably 1-50 nM, more preferably 1-20 nM, such as for example 8 nM. Exemplary concentration ranges of glucose include 10-100 mM, more preferably 10-50 mM, more preferably 10-25 mM, such as for example 25 mM.

According to one embodiment, the cells are redifferentiated by:

(a) incubating adult islet beta cells in a culture medium comprising nicotinamide, exendin-4, activin A and 10-50 mM glucose, the culture medium being devoid of serum; and subsequently

(b) incubating the adult islet beta cells in an additional culture medium comprising nicotinamide, exendin-4, and 0.5-10 mM glucose. The medium of step (b) is preferably devoid of activin A.

For step B, exemplary concentration ranges of glucose include 0.5-10 mM, more preferably 1-10 mM, more preferably 2.5-7.5 mM, such as for example 5.6 mM.

The cells are cultured in the above described mediums for at least one day, two days, 3 days, four days, five days, six days, seven days, eight days, nine days or ten days.

According to a particular embodiment, the cells are cultured in the above described media for 2-14 days, more preferably 2-10 days and even more preferably 2-8 days.

The culturing the above described culture media may be carried out prior to the down-regulation of ZEB-1/SOX-2/SOX-6, concomitant with the down-regulation of ZEB-1/SOX-2/SOX-6 and/or following the down-regulation of ZEB-1/SOX-2/SOX-6.

Together with down-regulation of ZEB-1/SOX-2/SOX-6 and optionally culturing the cells in a medium comprising differentiation factors under conditions to allow differentiation of the cells into insulin producing cells, the present inventors further contemplate downregulation of the protein Hairy and Enhancer of Split 1 (HES1; NM_{—}005524, NP_{—}005515).

Down-regulation of HES1 may be effected using any of the methods described herein above. Further information about downregulation of HES1 can be obtained from WO2009/078012, the contents of which are incorporated herein by reference.

According to another aspect of the present invention there is provided an isolated population of cells, comprising a heterologous polynucleotide which down-regulates a polypeptide, wherein the RNA transcript encoding the polypeptide is targeted by miRNA-200c, the polypeptide being selected from the group consisting of ZEB-1, SOX-2 and SOX-6, wherein the cells secrete insulin.

Since the redifferentiated cells of the present invention store and secrete insulin in a glucose responsive manner, they may be used for treating a disease which is associated with insulin deficiency such as diabetes.

Thus, according to another aspect of the present invention there is provided a method of treating diabetes in a subject, the method comprising transplanting a therapeutically effective amount of the population of ex-vivo expanded and re-differentiated adult islet beta cells of the present invention into the subject, thereby treating diabetes.

As used herein “diabetes” refers to a disease resulting either from an absolute deficiency of insulin (type 1 diabetes) due to a defect in the biosynthesis or production of insulin, or a relative deficiency of insulin in the presence of insulin resistance (type 2 diabetes), i.e., impaired insulin action, in an organism. The diabetic patient thus has absolute or relative insulin deficiency, and displays, among other symptoms and signs, elevated blood glucose concentration, presence of glucose in the urine and excessive discharge of urine.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

As used herein, “transplanting” refers to providing the redifferentiated adult islet beta cells of the present invention, using any suitable route. Typically, beta cell therapy is effected by injection using a catheter into the portal vein of the liver, although other methods of administration are envisaged.

As mentioned hereinabove, the adult islet beta cells of the present invention can be derived from either autologous sources or from allogeneic sources such as human cadavers or donors. Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

The cells of the present invention may be transplanted to a human subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the adult islet beta cells of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically.

Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Pharmaceutical compositions suitable for use in context of the present invention include compositions wherein the active ingredients are contained in an amount effective to achieve the intended purpose. More specifically, a therapeutically effective amount means an amount of active ingredients (insulin producing cells) effective to prevent, alleviate or ameliorate symptoms of a disorder (e.g., diabetes) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated from animal models (e.g. STZ diabetic mice) to achieve a desired concentration or titer. Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide cell numbers sufficient to induce normoglycemia (minimal effective concentration, MEC).

The MEC will vary for each preparation, but can be estimated from in vitro data.

Dosages necessary to achieve the MEC will depend on individual characteristics and route of administration. Detection assays can be used to determine plasma concentrations.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as further detailed above.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Cell Culture:

Human islets were received 2-4 days following isolation from individual donors.

Islets were dissociated into single cells and cultured in CMRL 1066 medium containing 5.6 mM D-glucose and supplemented with 10% FCS (HyClone), 100 U/ml penicillin, 100 μg/ml streptomycin, 100 μg/ml gentamycin, and 5 μg/ml amphotericine B (Biological Industries). Culture medium was changed every 3-4 days. Once confluency was reached (day 7-10 in each passage), cells were detached by trypsin (Versene) and reseeded in a 1:2 ratio. For redifferentiation, expanded cells in passages 5-7 were trypsinized and seeded in ultra-low attachment plates with Redifferentiation Cocktail (RC) for 4-8 days as previously described [Russ H A, Sintov E, Anker-Kitai L, Friedman O, Lenz A, Toren G, Farhy C, Pasmanik-Chor M, Oron-Karni V, Ravassard P, Efrat S. Insulin-producing cells generated from dedifferentiated human pancreatic beta cells expanded in vitro. PLoS One 2011; 6 (9):e25566]. The medium was replaced every two days.

A first redifferentiation cocktail (RC1) treatment consisted of 4 days in CMRL, 1% BSA, 25 mM D-glucose, B27 supplement (Stem Cell Technologies), ITS (Gibco-Invitrogen), 10 mM nicotinamide (Sigma), 8 nM exendin-4 (Acris), and 8 nM activin A (Peprotech). Antibiotics included penicillin and streptomycin.

A second redifferentiation cocktail (RC2) treatment consisted of 6 days in CMRL supplemented with 1% BSA, 25 mM D-glucose, N2 and B27 supplements (Stem Cell Technologies), ITS (Gibco-Invitrogen), 10 mM nicotinamide (Sigma), 8 nM exendin-4 (Acris), and 8 nM activin A (Peprotech), followed by 2 days in the same medium lacking activin A and containing a reduced glucose concentration (5.6 mM). Antibiotics used were penicillin and streptomycin.

Virus Production, Cell Infection and Cell Sorting:

RIP-Cre/ER and pTrip-loxP-NEO-STOP-loxP-eGFP lentiviruses [Russ H A, et al., PLoS One 2009; 4 (7):e6417] were used for lineage tracing. ZEB and scrambled control shRNA lentiviruses (TRCN-17563 and -17566 for ZEB1, and TRCN-13528 and -13530 for ZEB2) were obtained from Sigma-Aldrich. Virus production, cell infection, and tamoxifen treatment were previously described [Russ H A, et al. PLoS One 2009; 4 (7):e6417]. For ZEB inhibition, cells at passages 4-6 were infected at MOI 9:1 in CMRL 1066 medium containing 8 μg/ml polybrene (Sigma-Aldrich) overnight. eGFP-labeled cells were sorted using a FACS Aria sorter (BD Biosciences) as described [15].

qPCR Analysis:

Total RNA was extracted using ZR RNA MiniPrep™ kit (Zymo Research) or TRIzol (Sigma-Aldrich) including a DNase (Thermo Scientific) treatment step to remove genomic DNA. cDNA was prepared using High Capacity cDNA RT Kit (Applied Biosystems). qPCR was carried out using ABsolute blue qPCR Mix (Thermo Scientific) or FastStart Universal Probe Library Master Mix (Roche) in a 7300 real-time PCR instrument (Applied Biosystems). The results were normalized to transcripts of TATA-box-binding protein (TBP) and human large ribosomal protein (RPLPO), or glyceraldehyde-3-phosphate dehydrogenase (GAPDH). Table 1 lists primer sequences, designed for Universal Probe Library, a probe set of human RT-qPCR assays (Roche). All reactions were performed with annealing at 60′C for 40 cycles. For undetectable transcripts, the cycle number was set to 40 for comparisons.

TABLE 1

| Gene | Sense primer | Antisense primer |
|---|---|---|
| ACTA2 | GCTTTCAGCTTCCCTGAACA | GGAGCTGCTTCACAGGATTC |
| | SEQ ID NO: 1 | SEQ ID NO: 2 |
| CDH1 | GCCGAGAGCTACACGTTCA | GACCGGTGCAATCTTCAAA |
| | SEQ ID NO: 3 | SEQ ID NO: 4 |
| CDH2 | CTCCATGTGCCGGATAGC | CGATTTCACCAGAAGCCTCT |
| | SEQ ID NO: 5 | AC SEQ ID NO: 6 |
| CDKN1A | CCGAAGTCAGTTCCTTGTGG | CATGGGTTCTGACGGACAT |
| | SEQ ID NO: 7 | SEQ ID NO: 8 |
| CDKN1B | TTTGACTTGCATGAAGAGAA | AGCTGTCTCTGAAAGGGACA |
| | GC SEQ ID NO: 9 | TT SEQ ID NO: 10 |
| CETN2 | AGGATGAGTTCTTTAGCTTT | AATAGGCTGCCTTCAACACAA |
| | GCTT SEQ ID NO: 11 | SEQ ID NO: 12 |
| CLDN1 | CCTATGACCCCAGTCAATGC | ACAGCAAAGTAGGGCACCTC |
| | SEQ ID NO: 13 | SEQ ID NO: 14 |
| F11R | GCAGCCGTCCTTGTAACC | GGCTGGCTGTAAATCACCTT |
| | SEQ ID NO: 15 | SEQ ID NO: 16 |
| GCG | GTACAAGGCAGCTGGCAAC | TGGGAAGCTGAGAATGATCTG |
| | SEQ ID NO: 17 | SEQ ID NO: 18 |
| HLXB9 | TGCCTAAGATGCCCGACTT | AGCTGCTGGCTGGTGAAG |
| | SEQ ID NO: 19 | SEQ ID NO: 20 |
| IAPP | TTACCAAATTGTAGAGGCTT | CCCTGCCTCTATACACTCAC |
| | TCG SEQ ID NO: 21 | TACC SEQ ID NO: 22 |
| INADL | ACCCCTCGGTCTTAGGAAAA | TTGCTGATCTCTGCCGTGT |
| | SEQ ID NO: 23 | SEQ ID NO: 24 |
| INS | AGGCTTCTTCTACACACCCA | CACAATGCCACGCTTCTG |
| | AG SEQ ID NO: 25 | SEQ ID NO: 26 |
| ITGA3 | CAACCAGGATGGATTTCAGG | CAGTCCCAGCTTCTCTCCAT |
| | SEQ ID NO: 27 | SEQ ID NO: 28 |
| LMOD1 | GGAAGATGGGAGACAAAGTC | ACTGAAGCAGTTTGGGCACT |
| | C SEQ ID NO: 29 | SEQ ID NO: 30 |
| MAFA | AGCGAGAAGTGCCAACTCC | TTGTACAGGTCCCGCTCTTT |
| | SEQ ID NO: 31 | SEQ ID NO: 32 |
| MTOR | AGCACCATCAACCTCCAAAA | GGCGATGATGAGTCCTTCAG |
| | SEQ ID NO: 33 | SEQ ID NO: 34 |
| NEUROD1 | CTGCTCAGGACCTACTAACA | GTCCAGCTTGGAGGACCTT |
| | ACAA SEQ ID NO: 35 | SEQ ID NO: 36 |
| NKX2-2 | CGAGGGCCTTCAGTACTCC | GGGGACTTGGAGCTTGAGT |
| | SEQ ID NO: 37 | SEQ ID NO: 38 |
| NKX6-1 | CGTTGGGGATGACAGAGAGT | CGAGTCCTGCTTCTTCTTGG |
| | SEQ ID NO: 39 | SEQ ID NO: 40 |
| OCLN | AGGAACCGAGAGCCAGGT | TGAGCAATGCCCTTTAGCTT |
| | SEQ ID NO: 41 | SEQ ID NO: 42 |
| PPY | TCTAGTGCCCATTTACTCTG | GCAGGTGGACAGGAGCAG |
| | GAC SEQ ID NO: 43 | SEQ ID NO: 44 |
| PDX1 | CACATCCCTGCCCTCCTAC | GAAGAGCCGGCTTCTCTAAAC |
| | SEQ ID NO: 45 | SEQ ID NO: 46 |
| SST | ACCCCAGACTCCGTCAGTTT | ACAGCAGCTCTGCCAAGAAG |
| | SEQ ID NO: 47 | SEQ ID NO: 48 |
| SOX2 | GGGGGAATGGACCTTGTATA | GCAAAGCTCCTACCGTACCA |
| | G SEQ ID NO: 49 | SEQ ID NO: 50 |
| SOX6 | AAAGGCAATTTACCAGTGAT | CAAATGGAGAGGTGGCTTG |
| | TTCT SEQ ID NO: 51 | SEQ ID NO: 52 |
| TMEM2 | TCACTCTTCTTCCCTGTTCT | ACCCATGTGCAGGAACTGA |
| | GA SEQ ID NO: 53 | SEQ ID NO: 54 |
| VCL | GGAGGTGATTAACCAGCCAA | AATGATGTCATTGCCCTTGC |
| | T SEQ ID NO: 55 | SEQ ID NO: 56 |
| ZEB1 | GCCAACAGACCAGACAGTGT | TCTTGCCCTTCCTTTCCTG |
| | T SEQ ID NO: 57 | SEQ ID NO: 58 |
| ZEB2 | AAGCCAGGGACAGATCAGC | CCACACTCTGTGCATTTGAA |
| | SEQ ID NO: 59 | CT SEQ ID NO: 60 |

cDNA for miRNA analyses was prepared and analyzed using Taqman® MicroRNA Assay kit (Applied Biosystems) according to the manufacturer, with primers listed in Table 2.

TABLE 2

| Gene | Sequence | Assay no. |
|---|---|---|
| hsa-miR- | UAACACUG | 000502 |
| 200a | UCUGGUA | |
| | ACGAUGU; SEQ ID | |
| | NO: 61 | |
| hsa-miR- | UAAUACUG | 002251 |
| 200b | CCUGGUAA | |
| | UGAUGA; SEQ ID | |
| | NO: 62 | |
| hsa-miR- | UAAUACUG | 002300 |
| 200c | CCGGGUAA | |
| | UGAUGGA; SEQ ID | |
| | NO: 63 | |
| hsa-miR-24 | UGGCUCAG | 000402 |
| | UUCAGCAG | |
| | GAACAG; SEQ ID | |
| | NO: 64 | |
| U6 snRNA | TGCTCGCTT | 001973 |
| | CGGCAGCA | |
| | CATATACT | |
| | AAAATTGGA | |
| | ACGATACAG | |
| | AGAAGATTA | |
| | GCATGGCCC | |
| | CTGCGCAAG | |
| | GATGACACG | |
| | CAAATTCGT | |
| | GAAGCGTTC | |
| | CATATTTT; | |
| | SEQ ID NO: 65 | |

Immunofluorescence and Cell Proliferation Analyses:

Cells were trypsinyzed, spotted on slides using a Shandon Cytospin4 centrifuge (Thermo Scientific), and fixed for 15 min at RT in 4% paraformaldehyde (PFA). Samples were blocked for 30 min in PBS containing 1% BSA, 5% fetal goat serum (FGS) and 0.2% saponin (blocking buffer). Samples were then incubated overnight at 4° C. with primary antibodies diluted in blocking buffer as follows: rat anti-human C-peptide (1:1000; BCBC); mouse anti-human PDX1 (1:500; R&D); mouse anti-NKX2.2 (1:1000; Hybridoma Bank); mouse anti-N10(6.1 (1:1000; Hybridoma Bank); rabbit anti-eGFP (1:1000; Life Technologies), and mouse anti-Ki67 (1:200; Zymed) for cell proliferation analyses. Slides were washed in PBS with 0.1% Tween 20 (Sigma) and incubated with Alexa fluorophore-conjugated secondary antibodies (1:1000; Life Technologies). Nuclei were stained with DAPI or DRAQ5 (Abcam). Images were taken using a Leica SP5 confocal microscope.

Cell Apoptosis Analyses:

Apoptotic cells were detected by TUNEL staining using the In Situ Cell Death Detection Kit (Roche).

Immunoblotting:

Total protein was extracted from cells by incubation with a lysis buffer containing 0.5% NP-40 and protease inhibitor cocktail for 10 min 20-25 μg protein were electrophoresized on SDS-PAGE and electroblotted onto Immobilon-P 0.45-μm membrane (Millipore), followed by incubation overnight at 4° C. with rabbit anti-ZEB1 (1:1000; Cell Signaling) or mouse anti-ZEB2 (1:500; Abnova). Mouse anti-HSC70 (1:1000; Santa Cruz Biotechnology) was used to monitor loading. The bound antibody was visualized with the appropriate horseradish peroxidase-conjugated anti-IgG (Jackson Immunoresearch) and SuperSignal West Pico chemiluminescent substrate (Pierce). Quantification was done using TINA 2.0 software.

cDNA Microarray Analysis:

Hybridization to Affymetrix GeneChip Human Gene 1.1 ST Arrays, washing, and scanning were performed according to the manufacturer (Affymetrix). Data analysis was performed on CEL files using Partek Genomics Suite™ (Partek Inc.). Data were normalized with the multi-average method. Batch effect removal was applied for the different samples, followed by one-way ANOVA. Clustering analysis was performed by Partek Genomics Suite software with Pearson's dissimilarity correlation by average linkage methods.

Human C-Peptide and Cell Transplantation Analyses:

Cells were pre-incubated for 1 hour in Krebs-Ringer buffer (KRB), followed by incubation for 2 hours in KRB containing 0.5 mM 1-isobutyl 3-methylxanthine (IBMX) and 16.7 mM glucose. C-peptide content was determined in acidic alcohol cell extract. Human C-peptide was quantified using an ultrasensitive ELISA kit (Mercodia, Uppsala, Sweden; sensitivity 1.5 pmol/L; crossreactivity with insulin and proinsulin 0.0006% and 1.8%, respectively) according to the manufacturer's protocol. 1-2×10⁶ cells were injected with a 30-gauge needle under the kidney capsule of >12 week-old nonobese diabetic severe combined immunodeficient IL2Rγ^{-/-} (NOD-SCID-gamma, NSG) mice. Human C-peptide was quantified in serum samples obtained from the facial vein. Serum obtained from untreated mice was used as control.

Results

Induction of ZEB Expression During Islet Cell Dedifferentiation.

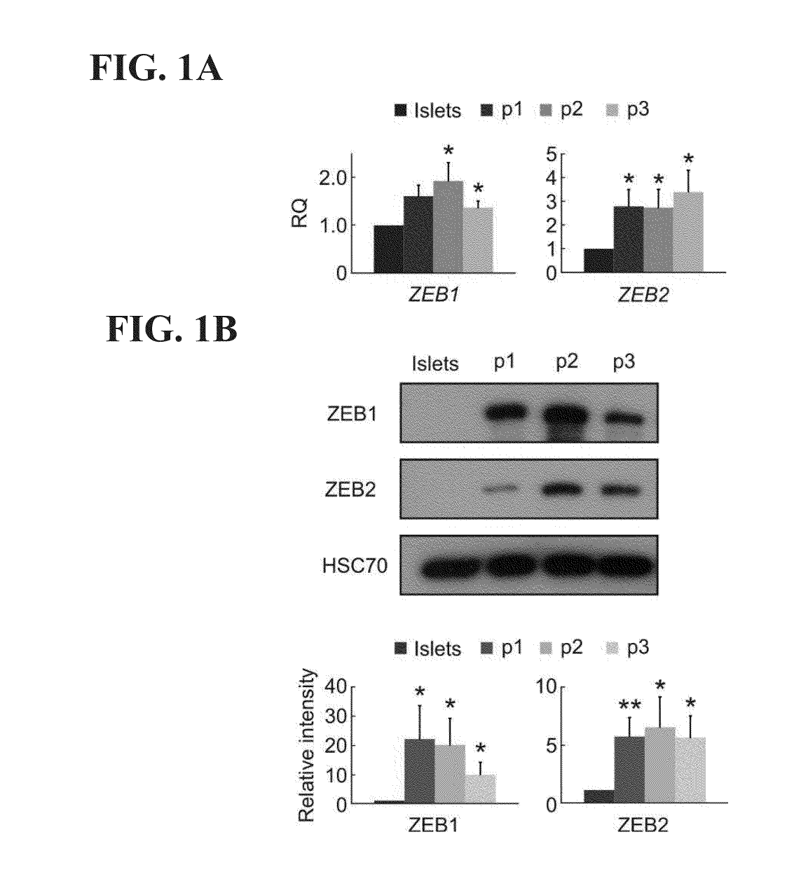

ZEB1 and ZEB2 transcripts were significantly upregulated in islet cells during the first 3 weeks of culture, as revealed by qPCR analyses (FIG. 1A) Immunoblotting revealed that both ZEB1 and ZEB2 were upregulated during the first week of culture, and their high levels were maintained thereafter during cell propagation (FIG. 1B).

ZEB1 Inhibition Induces Insulin Expression and Reduces BCD Cell Proliferation.

To evaluate the possibility for reversing the effects induced by ZEB proteins in cells which underwent EMT, shRNA was employed to block ZEB expression. Infection of expanded islet cells with two different ZEB1 shRNA lentiviruses reduced ZEB1 protein levels by up to 85±5% (FIG. 2A), while significantly elevating insulin transcript levels, relative to cells infected with a control shRNA (FIGS. 2B-2C). Among the two ZEB1 shRNAs, shRNA#1 was chosen for further experiments due to its higher efficiency. The levels of insulin transcripts were inversely proportional to the levels of ZEB1 transcripts, which were a function of the MOI of the ZEB1 shRNA virus (FIG. 2B). ZEB2 shRNA reduced ZEB2 protein levels by up to 65±40%. However, subsequent analyses revealed that ZEB2 inhibition did not significantly affect INS transcript levels. Therefore, further detailed analyses focused primarily on ZEB1 manipulation.

To determine the effect of ZEB1 inhibition on BCD cell replication, cells dissociated from isolated human islets were infected with two lentiviruses, one expressing Cre recombinase under control of the insulin promoter and the other, a reporter cassette with the structure cytomegalovirus (CMV) promoter-loxP-Stop-loxP-eGFP. In this system, eGFP expression is blocked by a loxP-flanked DNA fragment. Removal of the block specifically in β-cells activates eGFP expression during the initial days of culture, when the insulin promoter is still expressed, resulting in labeling of about 50% of β-cells. Labeled cells were then expanded, transduced with shRNA vectors, and stained for Ki67. The fraction of Ki67⁺ cells among eGFP-labelled BCD cells was significantly reduced about 3-fold following ZEB1 shRNA treatment, compared to control shRNA (FIG. 2D). To evaluate cell mortality rates 8-10 days following shRNA lentivirus infection, cells were analyzed by TUNEL assay. The ZEB1 shRNA treatment did not result in a significant increase in apoptosis, compared with control shRNA (0.43±0.12% and 0.47±0.25% TUNEL-positive cells for control and ZEB1 shRNAs, respectively, compared with 0.13±0.15% for uninfected cells).

Redifferentiation of BCD Cells Induced by ZEB1 Inhibition.

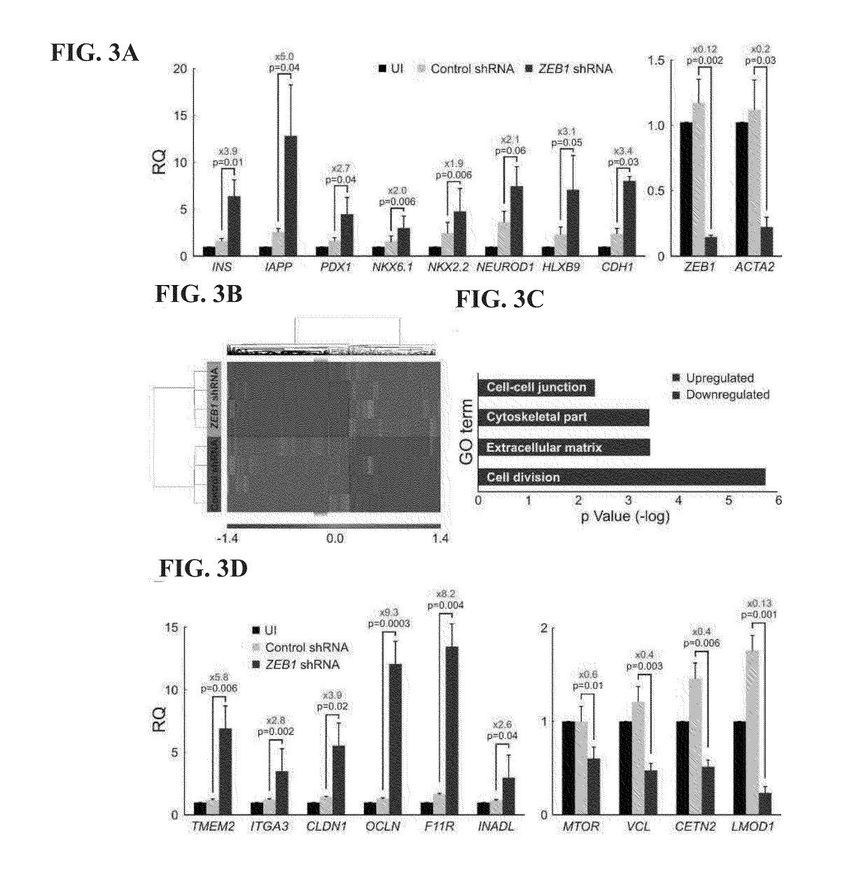

Previous findings demonstrated that restoration of β-cell gene expression in expanded islet cells represents BCD cell redifferentiation, rather than de-novo differentiation of non-BCD cells^{18,20,21}. To determine whether the observed changes induced in expanded islet cells by ZEB1 inhibition were due primarily to BCD cell redifferentiation, islet cells were labeled with the lineage tracing vectors, and BCD cells were sorted at passages 2-3 to 85-90% purity. ZEB1 inhibition in these cells led to a significant increase in transcripts encoding insulin, IAPP, and β-cell transcription factors, relative to the control shRNA, which was comparable to the increase observed in mixed islet cell cultures (FIG. 3A, left). CDH1 transcripts encoding the epithelial marker E-cadherin were upregulated 3.4-fold, while those for the mesenchymal marker ACTA2 (αSMA) were downregulated 5-fold, compared with the control shRNA, indicating the occurrence of MET (FIG. 3A, right). In contrast to INS transcripts, no significant increase was observed in transcripts encoding other islet hormones.

To assess global changes in gene expression following ZEB1 inhibition, cDNA microarray analyses were performed on RNA extracted from sorted BCD cells treated with ZEB1 or control shRNAs. A total of 380 genes were upregulated, while 287 genes were downregulated (fold change >1.5; pFDR<0.05) (FIG. 3B).

Table 3 lists the genes with the largest changes in expression.

TABLE 3

| | Gene | | | Fold- |
|---|---|---|---|---|
| | Symbol | Gene Assignment | p-Value | Change |
| Up- | LINGO2 | Leucine rich repeat and Ig | 2.14E−05 | 5.33 |
| reg- | | domain containing 2 | | |
| ulated | SPP1 | Secreted phosphoprotein 1 | 0.001471 | 5.11 |
| | TMEM2 | Transmembrane protein 2 | 4.01E−05 | 3.40 |
| | ACTBL2 | Actin, beta-like 2 | 1.04E−05 | 3.34 |
| | MIR503 | microRNA 503 | 3.97E−05 | 3.28 |
| | HIST2H4B | Histone cluster 2, H4b | 4.85E−06 | 3.18 |
| | MT1F | Metallothionein 1F | 0.000549 | 3.14 |
| | SLITRK6 | SLIT and NTRK-like | 9.85E−05 | 3.08 |
| | | family, member 6 | | |
| | PRR15 | Proline rich 15 | 2.45E−05 | 3.06 |
| | ABCC3 | ATP-binding cassette, | 1.15E−09 | 2.89 |
| | | sub-family C | | |
| | | (CFTR/MRP), member 3 | | |
| Down- | SCRG1 | Stimulator of | 3.19E−06 | −2.87 |
| reg- | | chondrogenesis 1 | | |
| ulated | KRTAP1-5 | Keratin associated protein | 7.75E−05 | −2.88 |
| | | 1- | | |
| | ZEB1 | Zinc finger E-box binding | 4.43E−07 | −2.91 |
| | | homeobox 1 | | |
| | SYT11 | Synaptotagmin XI | 1.01E−05 | −3.02 |
| | TNC | Tenascin C | 1.02E−05 | −3.02 |
| | MYOCD | Myocardin | 1.16E−05 | −3.06 |
| | LOXL4 | Lysyl oxidase-like | 0.000216 | −3.10 |
| | WRB | Tryptophan rich basic | 1.06E−06 | −3.16 |
| | | protein | | |
| | NTN4 | Netrin 4 | 8.53E−06 | −3.16 |
| | LMOD1 | Leiomodin 1 (smooth | 5.7E−05 | −3.43 |
| | | muscle) | | |

DAVID functional annotation revealed significant changes in 1291 downregulated genes (fold change >1.25; pFDR<0.05) sharing the annotation terms cell division, extracellular matrix, and cytoskeletal part, consistent with the effects of ZEB1 on cell proliferation and EMT (FIG. 3C). 1327 upregulated genes (fold change >1.25; pFDR<0.05) shared the cell-cell junction ontology term, implying a restoration of epithelial phenotype²⁸ (see Table4 for a full list of all significant GO terms).

TABLE 4

| | GO term | | | |
|---|---|---|---|---|
| | category | | # of | |
| | number | GO term | genes | p-Value |
| Up- | GO: 0045449 | regulation of transcription | 216 | 1.01E−08 |
| reg- | GO: 0015837 | amine transport | 20 | 4.97E−05 |
| ulated | GO: 0006865 | amino acid transport | 17 | 5.68E−05 |
| | GO: 0046942 | carboxylic acid transport | 21 | 3.42E−04 |
| | GO: 0015697 | quaternary ammonium | 6 | 3.74E−04 |
| | | group transport | | |
| | GO: 0015849 | organic acid transport | 21 | 3.74E−04 |
| | GO: 0031497 | chromatin assembly | 13 | 4.48E−03 |
| | GO: 0015804 | neutral amino acid | 6 | 6.20E−03 |
| | | transport | | |
| | GO: 0015695 | organic cation transport | 6 | 9.31E−03 |
| | GO: 0035195 | gene silencing by miRNA | 5 | 1.49E−02 |
| | GO: 0045941 | positive regulation of | 45 | 2.88E−02 |
| | | transcription | | |
| | GO: 0034329 | cell junction assembly | 7 | 3.03E−02 |
| | GO: 0035194 | posttranscriptional gene | 5 | 3.11E−02 |
| | | silencing by RNA | | |
| | GO: 0016441 | posttranscriptional gene | 5 | 3.11E−02 |
| | | silencing | | |
| | GO: 0010608 | posttranscriptional | 20 | 3.96E−02 |
| | | regulation of gene | | |
| | | expression | | |
| | GO: 0005911 | cell-cell junction | 20 | 4.56E−03 |
| | GO: 0043296 | apical junction complex | 13 | 5.01E−03 |
| | GO: 0008270 | zinc ion binding | 195 | 5.26E−08 |
| | GO: 0046914 | transition metal ion | 224 | 1.88E−07 |
| | | binding | | |
| | GO: 0043167 | ion binding | 316 | 3.00E−07 |
| | GO: 0046872 | metal ion binding | 309 | 3.87E−07 |
| | GO: 0043169 | cation binding | 310 | 6.95E−07 |
| | GO: 0015175 | neutral amino acid | 7 | 1.05E−03 |
| | | transmembrane transporter | | |
| | | activity | | |
| | GO: 0015294 | solute:cation symporter | 15 | 1.32E−03 |
| | | activity | | |
| | GO: 0015171 | amino acid transmembrane | 11 | 2.20E−03 |
| | | transporter activity | | |
| | GO: 0051119 | sugar transmembrane | 7 | 3.46E−03 |
| | | transporter activity | | |
| | GO: 0005351 | sugar:hydrogen symporter | 5 | 1.24E−02 |
| | | activity | | |
| | GO: 0005402 | cation:sugar symporter | 5 | 1.24E−02 |
| | | activity | | |
| | GO: 0015179 | L-amino acid | 7 | 2.28E−02 |
| | | transmembrane transporter | | |
| | | activity | | |
| | GO: 0035257 | nuclear hormone receptor | 10 | 3.71E−02 |
| | | binding | | |
| Down- | GO: 0031012 | extracellular matrix | 42 | 3.53E−04 |
| reg- | GO: 0044430 | cytoskeletal part | 93 | 3.70E−04 |
| ulated | GO: 0044421 | extracellular region part | 93 | 4.87E−04 |
| | GO: 0015630 | microtubule cytoskeleton | 59 | 5.59E−04 |
| | GO: 0043292 | contractile fiber | 19 | 1.40E−03 |
| | GO: 0005578 | proteinaceous extracellular | 35 | 6.73E−03 |
| | | matrix | | |
| | GO: 0015629 | actin cytoskeleton | 29 | 1.67E−02 |
| | GO: 0005876 | spindle microtubule | 6 | 4.44E−02 |
| | GO: 0005615 | extracellular space | 59 | 4.96E−02 |
| | GO: 0051301 | cell division | 42 | 1.73E−06 |
| | | activation of MAPK | | |
| | GO: 0000187 | activity | 16 | 1.77E−04 |
| | GO: 0022403 | cell cycle phase | 46 | 2.81E−04 |
| | GO: 0000278 | mitotic cell cycle | 41 | 6.71E−04 |
| | GO: 0007010 | cytoskeleton organization | 46 | 8.54E−04 |
| | GO: 0022402 | cell cycle process | 55 | 1.68E−03 |
| | GO: 0007155 | cell adhesion | 65 | 1.87E−03 |
| | GO: 0022610 | biological adhesion | 65 | 2.01E−03 |
| | GO: 0051302 | regulation of cell division | 10 | 2.47E−03 |
| | GO: 0051781 | positive regulation of cell | 9 | 2.70E−03 |
| | | division | | |
| | GO: 0034330 | cell junction organization | 11 | 2.86E−03 |
| | GO: 0007049 | cell cycle | 68 | 5.97E−03 |
| | GO: 0042127 | regulation of cell | 68 | 7.96E−03 |
| | | proliferation | | |
| | GO: 0051272 | positive regulation of cell | 14 | 8.91E−03 |
| | | motion | | |
| | GO: 0030335 | positive regulation of cell | 13 | 1.03E−02 |
| | | migration | | |
| | GO: 0051270 | regulation of cell motion | 22 | 1.10E−02 |
| | GO: 0007254 | JNK cascade | 10 | 1.15E−02 |
| | GO: 0008283 | cell proliferation | 41 | 1.20E−02 |
| | GO: 0030334 | regulation of cell | 19 | 2.15E−02 |
| | | migration | | |
| | GO: 0045104 | intermediate filament | 5 | 3.46E−02 |
| | | cytoskeleton organization | | |

To validate the microarray results, ten genes (Table 5) were analyzed by qPCR (FIG. 3D). Among the genes upregulated upon ZEB1 inhibition, OCLN, CLDN1, F11R and INADL encode epithelial tight junction proteins. F11R and INADL are direct ZEB1 targets, containing a ZEB1 binding site in their promoter region²⁸. Among downregulated genes, MTOR encodes a protein contributing to cell proliferation, while VCL encodes a protein involved in cell-cell and cell-matrix junctions.

TABLE 5

| | Gene | | | Fold- |
|---|---|---|---|---|
| | Symbol | Gene Assignment | p-Value | Change |
| Up- | TMEM2 | Transmembrane protein 2 | 4.0E−05 | 3.40 |
| regulated | CLDN1 | Claudin 1 | 2.5E−03 | 2.75 |
| | F11R | Fli receptor | 2.6E−05 | 2.23 |
| | ITGA3 | Integrin, alpha 3 (CD49C) | 3.1E−05 | 1.87 |
| | OCLN | Occludin | 4.6E−03 | 1.71 |
| | INADL | InaD-like (Drosophila) | 1.7E−03 | 1.65 |
| | | (PATJ) | | |
| Down- | MTOR | mechanistic target of | 3.6E−06 | −1.77 |
| regulated | | rapamycin | | |
| | | (serine/threonine kinase) | | |
| | VCL | Vinculin | 1.3E−06 | −1.83 |
| | CETN2 | Centrin, EF-hand protein, 2 | 3.3E−08 | −2.75 |
| | LMOD1 | Leiomodin 1 (smooth | 5.7E−05 | −3.43 |
| | | muscle) | | |

Human C-Peptide Release from Cells Treated with ZEB1 shRNA.

Expanded islet cells infected with ZEB1 or control shRNA were transplanted under the kidney capsule of NSG mice. A 2.4-fold increase in serum human C-peptide levels was observed in fed mice transplanted with ZEB1 shRNA-treated cells, compared to mice transplanted with control shRNA-treated cells (FIG. 4).

Synergistic Redifferentiation Effect of ZEB1 Inhibition and Soluble Factors.

Previous work has demonstrated the ability of BCD cells to undergo significant redifferentiation in response to a combination of soluble factors termed Redifferentiation Cocktail (RC) in serum-free medium¹⁸. ZEB1 silencing potentiated the effect of 8-day RC treatment on transcripts encoding the β-cell proteins insulin and IAPP, as well as the β-cell transcription factors PDX1, NEOROD1, NKX2.2, NKX6.1 and MAFA, 7-35-fold (FIG. 5A). ZEB1 silencing also potentiated the RC-induced downregulation of CDH2 transcripts, encoding N-cadherin, while CDH1 transcripts were significantly upregulated (FIG. 5B). In contrast to ZEB1 inhibition, ZEB2 shRNAs had only a marginal effect on INS transcript induction by RC, indicating that ZEB1 was the main effector in this system Immunostaining following a 4-day RC treatment revealed that ZEB1 shRNA induced a 2.4-fold increase in the fraction of C-peptide/PDX1 double-positive cells, compared to cells treated with RC and control shRNA (FIG. 5C). The fraction of C-peptide⁺ cells co-stained for the β-cell transcription factors NKX2.2 or NKX6.1 also increased significantly in ZEB1 shRNA-treated cells. Analysis of extracts of shRNA-infected cells following RC treatment revealed a 2-fold increase in C-peptide content in ZEB1 shRNA-treated cells, compared to cells treated with control shRNA (FIG. 5D). Considering the fraction of C-peptide⁺ cells in the cell population (FIG. 5C), the content achieved with the combined treatment represents an estimated 85% of C-peptide content of normal islet β cells. Glucose induced a 2.6-fold increase in insulin release in cells redifferentiated with the combined treatment, compared with a 1.3-fold increase in cells treated with RC and control shRNA (FIG. 5E). The higher secretory response to glucose induced by the combination of ZEB1 inhibition and RC treatment was due to both a lower basal secretion (33±6 ng/10⁶ cells per hour, compared to 49±48 ng/10⁶ cells treated with RC and control shRNA), as well as an elevated glucose-stimulated secretion (85±20 ng/10⁶ cells per hour, compared to 65±62 ng/10⁶ cells treated with RC and control shRNA), which are both manifestations of increased β-cell differentiation. The synergistic effect of RC and ZEB1 shRNA observed in cultures of mixed islet cells was reproduced in FACS-sorted BCD cells, as manifested by a β-fold increase in insulin transcript levels, compared to cells treated with RC and control shRNA (FIG. 5F). PDX1 and CDH1 transcripts were also upregulated by ZEB1 inhibition, compared to control shRNA. Co-staining of eGFP and C-peptide following ZEB1 inhibition and RC treatment revealed that the combined treatment led to redifferentiation of 60% of BCD cells (FIG. 5G). Given the cell labeling efficiency with eGFP (˜50%), the finding that >50% of C-peptide⁺ cells were eGFP⁺ suggests that BCD cells are the predominant source of insulin-expressing cells induced by the combined treatment, confirming our previous findings with RC treatment alone¹⁸. The 60% BCD cell redifferentiation efficiency is the highest achieved so far, compared with previously reported treatments^{18,20,21,29}.

ZEB1 Inhibition in BCD Cells is Associated with miR-200c Activation.

The occurrence of a double-negative feedback loop between the ZEB family of transcription factors and the miR-200 family of miRs in EMT and tumor invasion has been reported in recent years²³⁻²⁷. Using a miRNA microarray, the miR-200 family was identified as being among the miRNAs which were highly downregulated during BCD cell dedifferentiation²⁹. It was hypothesized that the ZEB1/miR-200 feedback loop may account in part for the ZEB1 effects on EMT, cell proliferation, and inhibition of insulin expression in expanded BCD cells. To investigate a possible involvement of miR-200, RNA from sorted eGFP-labeled BCD cells treated with ZEB1 shRNA was analyzed by qPCR for miR-200 expression. miR-200c was found to be significantly upregulated 3.3-fold following ZEB1 inhibition (FIG. 6A), while miR-200a and miR-200b were not significantly. Additionally, treatment of expanded islet cells with RC stimulated a similar increase in miR-200c levels (FIG. 6B), suggesting that miR-200c played a role in BCD cell redifferentiation induced by RC. The present inventors therefore studied the effect of miR-200c overexpression on redifferentiation of expanded islet cells using a retrovirus vector expressing pre-miR-200c. miR-200c levels in transduced cells were upregulated by 770±550 fold (mean±SE; n=3; p=0.03). Expression of SOX6 and SOX2, two transcription factors encoded by transcripts targeted by miR-200c^{30,31}, was downregulated in expanded islet cells overexpressing miR-200c by 43% and 37%, respectively (FIG. 6C). ZEB1 inhibition in expanded islet cells had a similar effect on these two miR-200c targets: SOX6 expression was significantly decreased by 33%, while SOX2 was downregulated by 15%, (FIG. 6D). SOX6 was shown to inhibit transactivation of the insulin gene by PDX1³², suggesting that its downregulation by miR-200c may stimulate insulin gene expression. Indeed, downregulation of SOX6 transcripts in expanded islet cells using SOX6 shRNA resulted in an increase in INS transcript levels similar to that induced by ZEB1 shRNA (FIG. 6E). SOX2 plays a role in stimulating cell proliferation and dedifferentiation, and has been shown to facilitate G1/S transition by suppression of p21^{Cip1} and p27^{Kip1}, two key cyclin/CDK inhibitors³³. In support of this mechanism, p21^{Cip1} (CDKN1A) and p27^{Kip1} (CDKN1B) transcripts were significantly elevated in ZEB1 shRNA-treated BCD cells, compared to cells treated with control shRNA (FIG. 6F). Furthermore, downregulation of SOX2 expression in expanded islet cells using SOX2 shRNA resulted in an increase in CDKN1B transcript levels similar to that induced by ZEB1 shRNA (FIG. 6G). Taken together, these findings suggest possible mechanistic links between ZEB1 downregulation, insulin gene expression, and growth arrest during BCD cell redifferentiation (FIG. 6H).

Discussion

ZEB1 and ZEB2 are activated in BCD cells in the early days of islet cell adaptation to growth in culture, and their elevated expression is maintained during cell expansion. To restore a β-cell-like phenotype in dedifferentiated BCD cells we employed a shRNA against ZEB1 transcripts Inhibition of ZEB1 expression by itself induced a phenotypic change in the dedifferentiated BCD cells, as manifested by upregulation of β-cell markers, acquisition of epithelial characteristics, and attenuation of proliferation. Furthermore, a synergistic effect was observed when ZEB1 shRNA treatment was combined with RC treatment, as manifested by higher levels of β-cell gene expression, and up to a 3.4-fold increase in the number of insulin-positive cells. Moreover, this effect was also achieved when ZEB1 was repressed in a sorted population of BCD cells, supporting the reported observation [Russ H A, et al., PLoS One 2011; 6 (9):e25566] that the main component of the redifferentiation effect observed in the total islet cell population is due to redifferentiation of BCD cells in the population. The results emphasize the key role of ZEB1 in EMT in BCD cells and confirm the importance of its down-regulation for BCD cell redifferentiation. This is also the first demonstration of redifferentiation of sorted BCD cells, indicating that non-BCD cells in the islet cell culture are not required for this process.

ZEB1 repression is significantly more effective in upregulating key β-cell genes and E-cadherin, compared with SLUG and ZEB2 silencing. Therefore, a more critical role can be attributed to ZEB1 involvement in EMT in adult human islet cells, compared to the other tested factors.

The effects of ZEB1 in BCD cells are likely mediated, at least in part, by the ZEB1/miR-200c feedback loop. This possibility is supported by the increase in miR-200c levels following ZEB1 inhibition. An increase in miR-200c levels upon RC treatment may also explain the synergistic effect of combined ZEB1 shRNA and RC treatments on BCD cell redifferentiation. Two miR-200c targets, SOX6 and SOX2, are suggested as possible mediators of the miR-200c effects. Direct inhibition of SOX6 expression by shRNA led to an increase in insulin transcript levels, which is comparable to that achieved with ZEB1 inhibition. Similarly, SOX2 shRNA upregulated CDKN1A and CDKN1B transcript levels to a similar extent as ZEB1 inhibition. Conceivably, additional elements could be involved in mediating the ZEB1 effects. The additive effect between ZEB1 and HES1 shRNAs suggests the existence of non-overlapping targets of ZEB1 and the NOTCH pathway in EMT in BCD cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Kroon E, Martinson L A, Kadoya K, Bang A G, Kelly O G, Eliazer S, Young H, Richardson M, Smart N G, Cunningham J, Agulnick A D, D'Amour K A, Carpenter M K, Baetge E E. Pancreatic endoderm derived from human embryonic stem cells generates glucose-responsive insulin-secreting cells in vivo. Nat Biotechnol 2008; 26:443-452.
2. Pagliuca F W, Millman J R, Gunter M, Segel M, Van Dervort A, Ryu J H, Peterson Q P, Greiner D, Melton D A. Generation of Functional Human Pancreatic beta Cells In Vitro. Cell 2014; 159:428-439.
3. Rezania A, Bruin J E, Arora P, Rubin A, Batushansky I, Asadi A, O'Dwyer S, Quiskamp N, Mojibian M, Albrecht T, Yang Y H, Johnson J D, Kieffer T J. Reversal of diabetes with insulin-producing cells derived in vitro from human pluripotent stem cells. Nat Biotechnol 2014; 32:1121-1133.
4. Zalzman M, Gupta S, Gin R K, Berkovich I, Sappal B S, Karnieli 0, Zern M A, Fleischer N, Efrat S. Reversal of hyperglycemia in mice by using human expandable insulin-producing cells differentiated from fetal liver progenitor cells. Proceedings of the National Academy of Sciences of the United States of America 2003; 100:7253-7258.
5. Ouziel-Yahalom L, Zalzman M, Anker-Kitai L, Knoller S, Bar Y, Glandt M, Herold K, Efrat S. Expansion and redifferentiation of adult human pancreatic islet cells. Biochemical and biophysical research communications 2006; 341:291-298.
6. Russ H A, Bar Y, Ravassard P, Efrat S. In vitro proliferation of cells derived from adult human beta-cells revealed by cell-lineage tracing. Diabetes 2008; 57:1575-1583.
7. Russ H A, Ravassard P, Ken-Conte J, Pattou F, Efrat S. Epithelial-mesenchymal transition in cells expanded in vitro from lineage-traced adult human pancreatic beta cells. PloS one 2009; 4:e6417.
8. Moustakas A, Heldin C H. Signaling networks guiding epithelial-mesenchymal transitions during embryogenesis and cancer progression. Cancer Sci 2007; 98:1512-1520.
9. Thiery J P, Acloque H, Huang R Y, Nieto M A. Epithelial-mesenchymal transitions in development and disease. Cell 2009; 139:871-890.
10. Katoh Y, Katoh M. Hedgehog signaling, epithelial-to-mesenchymal transition and miRNA (review). Int J Mol Med 2008; 22:271-275.
11. Valenta T, Hausmann G, Basler K. The many faces and functions of beta-catenin. The EMBO journal 2012; 31:2714-2736.
12. Wang Z, Li Y, Kong D, Sarkar F H. The role of Notch signaling pathway in epithelial-mesenchymal transition (EMT) during development and tumor aggressiveness. Curr Drug Targets 11:745-751.
13. Xu J, Lamouille S, Derynck R. TGF-beta-induced epithelial to mesenchymal transition. Cell Res 2009; 19:156-172.
14. Battle E, Sancho E, Franci C, Dominguez D, Monfar M, Baulida J, Garcia De Herreros A. The transcription factor snail is a repressor of E-cadherin gene expression in epithelial tumour cells. Nat Cell Biol 2000; 2:84-89.
15. Moreno-Bueno G, Cubillo E, Sarrio D, Peinado H, Rodriguez-Pinilla S M, Villa S, Bolos V, Jorda M, Fabra A, Portillo F, Palacios J, Cano A. Genetic profiling of epithelial cells expressing E-cadherin repressors reveals a distinct role for Snail, Slug, and E47 factors in epithelial-mesenchymal transition. Cancer Res 2006; 66:9543-9556.
16. Shirakihara T, Saitoh M, Miyazono K. Differential regulation of epithelial and mesenchymal markers by deltaEF1 proteins in epithelial mesenchymal transition induced by TGF-beta. Mol Biol Cell 2007; 18:3533-3544.
17. Bar-Nur O, Russ H A, Efrat S, Benvenisty N. Epigenetic memory and preferential lineage-specific differentiation in induced pluripotent stem cells derived from human pancreatic islet Beta cells. Cell Stem Cell 2011; 9:17-23.
18. Russ H A, Sintov E, Anker-Kitai L, Friedman 0, Lenz A, Toren G, Farhy C, Pasmanik-Chor M, Oron-Karni V, Ravassard P, Efrat S. Insulin-producing cells generated from dedifferentiated human pancreatic beta cells expanded in vitro. PloS one 2011; 6:e25566.
19. Bar Y, Russ H A, Knoller S, Ouziel-Yahalom L, Efrat S. HES-1 is involved in adaptation of adult human beta-cells to proliferation in vitro. Diabetes 2008; 57:2413-2420.
20. Lenz A, Toren-Haritan G, Efrat S. Redifferentiation of Adult Human beta Cells Expanded In Vitro by Inhibition of the WNT Pathway. PloS one 2014; 9:e112914.
21. Bar Y, Russ H A, Sintov E, Anker-Kitai L, Knoller S, Efrat S. Redifferentiation of expanded human pancreatic beta-cell-derived cells by inhibition of the NOTCH pathway. The Journal of biological chemistry 2012; 287:17269-17280.
22. Eger A, Aigner K, Sonderegger S, Dampier B, Oehler S, Schreiber M, Berx G, Cano A, Beug H, Foisner R. DeltaEF1 is a transcriptional repressor of E-cadherin and regulates epithelial plasticity in breast cancer cells. Oncogene 2005; 24:2375-2385.
23. Brabletz S, Brabletz T. The ZEB/miR-200 feedback loop—a motor of cellular plasticity in development and cancer? EMBO reports 2010; 11:670-677.
24. Burk U, Schubert J, Wellner U, Schmalhofer O, Vincan E, Spaderna S, Brabletz T. A reciprocal repression between ZEB1 and members of the miR-200 family promotes EMT and invasion in cancer cells. EMBO reports 2008; 9:582-589.
25. Gregory P A, Bert A G, Paterson E L, Barry S C, Tsykin A, Farshid G, Vadas M A, Khew-Goodall Y, Goodall G J. The miR-200 family and miR-205 regulate epithelial to mesenchymal transition by targeting ZEB1 and SIP1. Nat Cell Biol 2008; 10:593-601.
26. Korpal M, Lee E S, Hu G, Kang Y. The miR-200 family inhibits epithelial-mesenchymal transition and cancer cell migration by direct targeting of E-cadherin transcriptional repressors ZEB1 and ZEB2. The Journal of biological chemistry 2008; 283:14910-14914.
27. Park S M, Gaur A B, Lengyel E, Peter M E. The miR-200 family determines the epithelial phenotype of cancer cells by targeting the E-cadherin repressors ZEB1 and ZEB2. Genes & development 2008; 22:894-907.
28. Voorhoeve P M, le Sage C, Schrier M, Gillis A J, Stoop H, Nagel R, Liu Y P, van Duijse J, Drost J, Griekspoor A, Zlotorynski E, Yabuta N, De Vita G, Nojima H, Looijenga L H, Agami R. A genetic screen implicates miRNA-372 and miRNA-373 as oncogenes in testicular germ cell tumors. Cell 2006; 124:1169-1181.
29. Irizarry R A, Hobbs B, Collin F, Beazer-Barclay Y D, Antonellis K J, Scherf U, Speed T P. Exploration, normalization, and summaries of high density oligonucleotide array probe level data. Biostatistics 2003; 4:249-264.
30. Aigner K, Dampier B, Descovich L, Mikula M, Sultan A, Schreiber M, Mikulits W, Brabletz T, Strand D, Obrist P, Sommergruber W, Schweifer N, Wernitznig A, Beug H, Foisner R, Eger A. The transcription factor ZEB1 (deltaEF1) promotes tumour cell dedifferentiation by repressing master regulators of epithelial polarity. Oncogene 2007; 26:6979-6988.
31. Tatarkiewicz K, Garcia M, Omer A, Van Schilfgaarde R, Weir G C, De Vos P. C-peptide responses after meal challenge in mice transplanted with microencapsulated rat islets. Diabetologia 2001; 44:646-653.
32. Nathan G, Kredo-Russo S, Geiger T, Lenz A, Kaspi H, Hornstein E, Efrat S. miR-375 promotes redifferentiation of adult human β cells expanded in vitro. PloS one 2015; In press.
33. Melkman-Zehavi T, Oren R, Kredo-Russo S, Shapira T, Mandelbaum A D, Rivkin N, Nir T, Lennox K A, Behlke M A, Dor Y, Hornstein E. miRNAs control insulin content in pancreatic beta-cells via downregulation of transcriptional repressors. The EMBO journal 2011; 30:835-845.
34. Peng C, Li N, Ng Y K, Zhang J, Meier F, Theis F J, Merkenschlager M, Chen W, Wurst W, Prakash N. A unilateral negative feedback loop between miR-200 microRNAs and Sox2/E2F3 controls neural progenitor cell-cycle exit and differentiation. J Neurosci 2012; 32:13292-13308.
35. Iguchi H, Ikeda Y, Okamura M, Tanaka T, Urashima Y, Ohguchi H, Takayasu S, Kojima N, Iwasaki S, Ohashi R, Jiang S, Hasegawa G, Ioka R X, Magoori K, Sumi K, Maejima T, Uchida A, Naito M, Osborne T F, Yanagisawa M, Yamamoto T T, Kodama T, Sakai J. SOX6 attenuates glucose-stimulated insulin secretion by repressing PDX1 transcriptional activity and is down-regulated in hyperinsulinemic obese mice. The Journal of biological chemistry 2005; 280:37669-37680.
36. Herreros-Villanueva M, Zhang J S, Koenig A, Abel E V, Smyrk T C, Bamlet W R, de Narvajas A A, Gomez T S, Simeone D M, Bujanda L, Billadeau D D. SOX2 promotes dedifferentiation and imparts stem cell-like features to pancreatic cancer cells. Oncogenesis 2013; 2:e61.
37. Talchai C, Xuan S, Lin H V, Sussel L, Accili D. Pancreatic beta cell dedifferentiation as a mechanism of diabetic beta cell failure. Cell 2012; 150:1223-1234.
38. White M G, Marshall H L, Rigby R, Huang G C, Amer A, Booth T, White S, Shaw J A. Expression of mesenchymal and alpha-cell phenotypic markers in islet beta-cells in recently diagnosed diabetes. Diabetes care 2013; 36:3818-3820.

What is claimed is:

1. A method of ex-vivo increasing insulin content in progenitor cells which express Zinc Finger E-Box Binding Homeobox 1 (ZEB-1), comprising contacting the progenitor cells with an inhibitory agent directed against a polypeptide, wherein the RNA transcript encoding said polypeptide is targeted by miRNA-200c, said polypeptide being selected from the group consisting of ZEB-1, SOX-2 and SOX-6, thereby increasing insulin content in the progenitor cells.

2. The method of claim 1, wherein said progenitor cells are selected from the group consisting of dedifferentiated adult islet beta cells, mesenchymal stem cells and induced pluripotent stem cells dedifferentiated from beta cells.

3. The method of claim 1, wherein said inhibitory agent is a polynucleotide agent.

4. The method of claim 1, further comprising culturing the progenitor cells in a medium comprising nicotinamide, exendin-4, activin A and glucose, said culture medium being devoid of serum.

5. The method of claim 4, which said culturing is effected following or concomitant with said contacting.

6. The method of claim 4, comprising:
(a) exposing said progenitor cells to a culture medium comprising said nicotinamide, said exendin-4, said activin A, wherein said glucose is present at a concentration of 10-100 mM; and subsequently
(b) exposing said progenitor cells in an additional culture medium comprising said nicotinamide and said exendin-4, wherein said glucose is present at a concentration of 0.5-10 mM.

7. The method of claim 6, wherein said additional medium is devoid of activin A.

8. The method of claim 1, further comprising contacting the progenitor cells with an agent that down-regulates an activity and/or amount of HES-1.

9. The method of claim 2, wherein said dedifferentiated adult islet beta cells comprise induced pluripotent stem cells generated from beta cells.

10. The method of claim 2, wherein said dedifferentiated adult islet beta cells are generated by culturing said adult islet beta cells for at least 10 passages.

11. The method of claim 10, wherein said culturing is effected in CMRL medium.

12. An isolated population of cells, comprising a heterologous polynucleotide which down-regulates a polypeptide, wherein the RNA transcript encoding said polypeptide is targeted by miRNA-200c, said polypeptide being selected from the group consisting of ZEB-1, SOX-2 and SOX-6, wherein said cells secrete insulin.

13. An isolated population of cells generated according to the method of claim 1.

14. A method of treating diabetes in a subject, comprising transplanting a therapeutically effective amount of the population of adult islet beta cells of claim 12 into the subject, thereby treating diabetes.

15. The isolated population of cells of claim 12, being genetically modified to express a pharmaceutical agent.

16. The isolated population of cells of claim 12, being adult islet beta cells.

17. A pharmaceutical composition comprising as an active ingredient the population of cells of claim 12 and a pharmaceutically acceptable carrier.

18. A method of treating Diabetes in a subject in need thereof comprising administering to the subject a therapeutically effective amount of an inhibitory agent directed against a polypeptide, wherein the RNA transcript encoding said polypeptide is targeted by miRNA-200c, said polypeptide being selected from the group consisting of ZEB-1, SOX-2 and SOX-6, thereby treating the Diabetes.

19. The method of claim 18, wherein said inhibitory agent is a polynucleotide agent.

20. The method of claim 18, wherein said inhibitory agent is a small molecule agent.
